(19) United States
(12) Patent Application Publication
(10) Pub. No.: US 2022/0305491 A1
(43) Pub. Date: Sep. 29, 2022

(54) SCALABLE SYSTEMS AND METHODS FOR AUTOMATED BIOSYSTEM ENGINEERING

(71) Applicant: Mekonos Inc., San Francisco, CA (US)

(72) Inventors: Mark A. WEBB, Berkeley, CA (US); Marc S. CHOOLJIAN, Berkeley, CA (US); Lauren M. OTTO, Berkeley, CA (US)

(73) Assignee: Mekonos Inc., San Francisco, CA (US)

(21) Appl. No.: 17/657,039

(22) Filed: Mar. 29, 2022

Related U.S. Application Data
(60) Provisional application No. 63/167,554, filed on Mar. 29, 2021.

(51) Int. Cl. B01L 3/00; B81B 1/00

(52) U.S. Cl. CPC B01L 3/502761; B81B 1/006; B01L 3/502715; B81B 2201/057; B81B 2203/0127; B81B 2203/0315; B81B 2203/033; B81B 2203/04; B01L 2300/0645; B01L 2400/0424; B01L 2300/0819

(57) ABSTRACT

An integrated package comprising a lab-on-chip (LOC) is disclosed. The LOC includes at least one integrated device having a membrane portion having a membrane opening; the membrane portion having a first side and a second side, the first side opposite the second side, a MEMS portion disposed on the first side of the membrane portion, the MEMS portion having a sharp member disposed on an actuator stage within a MEMS cavity, and a fluidic portion disposed on the second side of the membrane portion, the fluidic portion having a fluidic cavity for flowing a fluid medium within the fluidic portion; and a fluidic cap forming a surface of the fluidic portion of the LOC, the fluidic cap having a fluidic inlet and a fluidic outlet. The method of operating the LOC includes power to the at least one integrated device to capture one or more particles for interrogation.

### CROSS-REFERENCE TO RELATED APPLICATIONS

The present application claims the benefit of U.S. Provisional Application No. 63/167,554, filed on Mar. 29, 2021, the contents of which are incorporated herein by reference as if set forth in full.

### BACKGROUND

Medical treatment approaches, such as gene therapy via cellular transformations, are promising treatment options for a number of diseases, including inherited disorders, some types of cancer, and certain viral infections. Although gene therapy is promising, at present it is an experimental treatment based on insertion of genetic materials (genes) into a patient's cells instead of using drugs or surgery. Since the technique centers around introduction of genetic materials (or any biological molecules) into living cells, it is inherently risky and challenging, particularly for the danger of causing damage to the cells. Current manufacturing approaches to gene therapy agents include, for example, an electroporation process where an application of a very high electric field to living cells to create transient openings in the membranes of the cells. The electric field is typically applied globally in a cuvette to a large number of cells, and neither the electrical field for a given cell, nor the amount of material that enters the cell is controlled for any individual cell. Therefore, there is a need for a novel system and technological platform that cause less damage on the cell membrane than the applied fields used for electroporation, while offering isolation of living cells for direct manipulation in a fluidic environment, for example, to insert genetic materials into the cells. The need for such novel system is required to advance a promising treatment technique such as gene therapy without harmful side effects present with the aforementioned approaches.

### SUMMARY

In accordance with various embodiments, an integrated device is provided. The integrated device includes a membrane portion having a membrane opening; the membrane portion having a first side and a second side, the first side opposite the second side; a MEMS portion disposed on the first side of the membrane portion, the MEMS portion having a sharp member disposed on an actuator stage within a MEMS cavity, the sharp member having a distal (or base) portion attached substantially perpendicular to the actuator stage; and a fluidic portion disposed on the second side of the membrane portion, the fluidic portion having a fluidic cavity for flowing a fluid medium within the fluidic portion, wherein the membrane opening provides access between the MEMS portion and the fluidic portion and is substantially aligned with a proximal portion of the sharp member, and in operation, the proximal portion of the sharp member moves across the membrane opening and into at least a portion of the fluidic cavity.

In accordance with various embodiments, an integrated package is provided. The integrated package includes a substrate; and a lab-on-chip (LOC) disposed on the substrate, the LOC comprising at least one integrated device comprising: a membrane portion having a membrane opening; the membrane portion having a first side and a second side, the first side opposite the second side, a MEMS portion disposed on the first side of the membrane portion, the MEMS portion having a sharp member disposed on an actuator stage within a MEMS cavity, and a fluidic portion disposed on the second side of the membrane portion, the fluidic portion having a fluidic cavity for flowing a fluid medium within the fluidic portion; and a fluidic cap forming a surface of the fluidic portion of the LOC, the fluidic cap having a fluidic inlet and a fluidic outlet.

In accordance with various embodiments, a method for operating an integrated package is provided. The method includes providing a power source; providing the integrated package comprising at least one integrated device comprising: a membrane portion having a membrane opening; the membrane portion having a first surface on a first side and a second side, the first side opposite the second side, a MEMS portion disposed on the first side of the membrane portion, the MEMS portion having a sharp member disposed on an actuator stage configured as an electrode and a pull-toward electrode disposed on the first surface of the membrane portion substantially parallel to the actuator stage, and a fluidic portion disposed on the second side of the membrane portion; supplying, via the power source, a voltage across the actuator stage and the pull-toward electrode; generating an electrostatic field between the actuator stage and the pull-toward electrode based on the supplied voltage; and actuating the sharp member to move across the membrane opening and into at least a portion of the fluidic cavity due to the generated electrostatic field between the actuator stage and the pull-toward electrode.

In accordance with various embodiments, a method for operating an integrated device is provided. The method includes providing a power source; providing the integrated device comprising: a membrane portion having a membrane opening; the membrane portion having a first side and a second side, wherein the second side of the membrane portion comprises one or more capture-site electrodes disposed thereon, a MEMS portion disposed on the first side of the membrane portion, and a fluidic portion disposed on the second side of the membrane portion, the fluidic portion comprising a fluidic cap forming a fluidic cavity in the fluidic portion, the fluidic cap having a surface thereon, at least one fluidic inlet, at least one fluidic outlet, and one or more counter-electrodes disposed on the surface of the fluidic cap across from the membrane opening; supplying, via the power source, an AC voltage across the one or more counter-electrodes and the one or more capture-site electrodes; and generating an electric field with a local maximum proximate the membrane opening.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1A illustrates an embodiment of an integrated device, in accordance with various embodiments;

FIG. 1B shows a schematic view of the integrated device of FIG. 1A;

FIG. 2A illustrates an embodiment of an integrated device packaged as a lab-on-chip system, in accordance with various embodiments;

FIG. 2B shows a schematic illustration of a process flow for packaging a lab-on-chip system, in accordance with various embodiments;

FIG. 3 shows a schematic view of an array of the example MEMS portion of an integrated device, according to various implementations;

FIG. 4 is a flow chart for a method of operating an integrated device, according to an illustrative implementation; and

FIG. 5 is a flow chart for another method of operating an integrated device, according to an illustrative implementation.

### DETAILED DESCRIPTION

There are several solutions to circumvent the aforementioned issues pertaining to broad application of the electric field used in electroporation. One non-limiting approach may include using a capture mechanism to isolate one or more living cells for direct manipulation, for example, to insert a material, particle, or molecules into the cell. In accordance with various embodiments, a capture mechanism may include, for example but not limited to a method based on dielectrophoresis (DEP). DEP is an electro-physical phenomenon that occurs when a polarizable particle, such as a biological molecule, vesicle, or cell, in a non-linear electric field experiences a force aligned with the electric field gradient. This occurs because one side of the particle experiences a larger dipole force than the other due to the variation in electric field across the particle. Based on the ability to trap and sort neutral particles, such as inorganic nanoparticles, or biological molecules in fluidic environments, DEP can be exploited, for example, for single-cell analyses in microfluidic-based applications, such as gene therapy, as discussed above. Using DEP in standard biochemical assays by, for example, applying DEP to isolate single cells for impedance or fluorescence characterization (or any non-contact evaluation technique) has been demonstrated in fluidic environments.

The technology described herein includes an integrated system that enables a safer approach to inserting genetic materials. The disclosed integrated system can include various components integrated into a system that can be configured to manipulate nano- or microscale materials at a nanoscale or cellular level in a fluidic and/or an applied electric field (non-linear or linear) environment. The disclosed integrated system can include, for example, a fluidics-based capture architecture, a micro-electro-mechanical-system (MEMS)-based sample interrogation architecture, a chemical system and methodology for molecular spatiotemporal control. Moreover, the technology disclosed herein includes packaging architectures that allow for functional integration of aforementioned architectures and elements into a lab-on-a-chip (LOC) system that can serve a wide variety of commercial and research purposes. In accordance with various embodiments, the disclosed technology can enable controllable introduction of genetic materials (biological molecules in general) and/or inorganic nanoparticles into living cells by mechanical means, while causing minimal or no damage to the cells so that a high percentage of the cells are unharmed. Example genetic materials can include, for example, genetic macromolecules as well as other molecule classes that include, for example, proteins, peptides, small molecules, protein complexes with RNAs or DNAs, and any combinations thereof. Significant advantages of a mechanical insertion approach include having a precise mechanical control of the insertion tool, in addition to controlling the precise amount of active molecules, for example, new genetic materials, that are to be transported by the mechanical tool.

As described herein, the term “MEMS actuator” or “actuator” is composed of the actuator stage and actuator arms and is the moveable device layer that is contained in the MEMS cavity. This part was formerly called the cantilever.

As described herein, the term “actuator arms” refers to the typically serpentine arms that connect the actuator stage to the rest of the device layer silicon, known herein as the extra-cavity device layer (because it resides outside of the MEMS cavity). The actuator arms were formerly called the serpentine arms.

As described herein, the term “actuator stage” refers to the moveable stage on which the sharp member(s) rests inside the MEMS cavity.

As described herein, the term “ball grid array (BGA)” refers to a surface mount for bonding of the chip to a PCB or chip carrier during packaging, consisting of an array of contact pads covered by beads of solder and flux.

As described herein, the term “BioMEMS” refers to MEMS used in biological applications, also sometimes referring more broadly to microfluidic systems with biological applications that do not necessarily contain solid mechanical elements (as in, “fluid mechanics”).

As described herein, the term “bonded” refers to an irreversible bond between the materials at hand. Bonded or bonding includes but is not limited to chip-to-chip bonding, wafer bonding, types of bonding commonly used in the device packaging field to describe plastic to plastic bonding.

As described herein, the term “capture” is defined as the attraction of a particle to a particular location or site from a bulk mixture or flow as used herein and can also be referred to herein as “trap” and “immobilize” but will further be denoted as “capture.”

As described herein, the term “capture site” refers to the general locale in which the dielectrophoretic capture force will drive a particle to and hold it in and is proximate to the opening in/through the membrane portion.

As described herein, the term “capture-site electrode(s)” refers to the DEP electrode(s) located near the capture site. In most common embodiments, these are the active electrodes (i.e., the electrodes carrying the driven DEP signal)

As described herein, the term “carrier fluid” refers to a liquid that acts as a solvent and/or medium of transport for particles and reagents of interest.

As described herein, the term “chemical system” refers to and include: sharp member surface chemistry, surface treatments for hydrophilicity modulation, anti-fouling, etc.

As described herein, the term “chip” is used to denote the core of the device architecture comprising the MEMS portion bonded to the membrane portion herein. “Chip” can also include other portions, such as the interposer portion, that are directly bonded to or fabricated as part of the MEMS and membrane portions or their respective wafers and dies.

As described herein, the term “control elements” refers to electrical systems that are used to close the control loop between device sensing, device actuation, and external input.

As described herein, the term “counter-electrode” refers to the common ground electrode positioned across the fluidic cavity from the capture site electrode(s).

As described herein, the term “dielectrophoresis (DEP)” refers to an electro-physical phenomenon that occurs when a polarizable substance, such as a biological molecule or a cell and generally referred to as a particle, in a non-linear electric field experiences a force in the electric field gradient as used herein.

As described herein, the term “DEP electrodes” refers to one or more electrodes (also referred to herein as the “capture site electrodes”) and the counter-electrode as used herein.

As described herein, the term “device layer” refers to the layer of material that comprises the actuator (the device) and the material in the same layer outside the MEMS cavity.

As described herein, the term “discrete abiotic systems” refers to things such as nanoparticles, lipid vesicles, emulsions, or other multiphase systems.

As described herein, the term “discrete biological systems” refers to things such as living cells but can include other biological systems.

As described herein, the term “electrical signal I/O” refers to the inputs and outputs of the electrical signal on the LOC.

As described herein, the term “events” are elements in operational workflows of the LOC. These events can be broadly characterized as fluid dynamic events, electrical signal events, mechanical events, sample events, biological events, chemical events, physical events, operator input events, or general runtime events.

As described herein, the term “extra-cavity device layer” is the portion of the MEMS device layer that is not in the MEMS cavity.

As described herein, the term “fluid I/O” refers to the inlets and outlets of the fluid on the LOC.

As described herein, the term “fluidic cap” refers to a subcomponent of the fluidic portion. In some embodiments, the fluidic cap is not bonded directly to the membrane portion.

As described herein, the term “fluidic cavity” refers to the region where the particle and its carrier fluid are found and are subjected to the electric field that generates the DEP capture force.

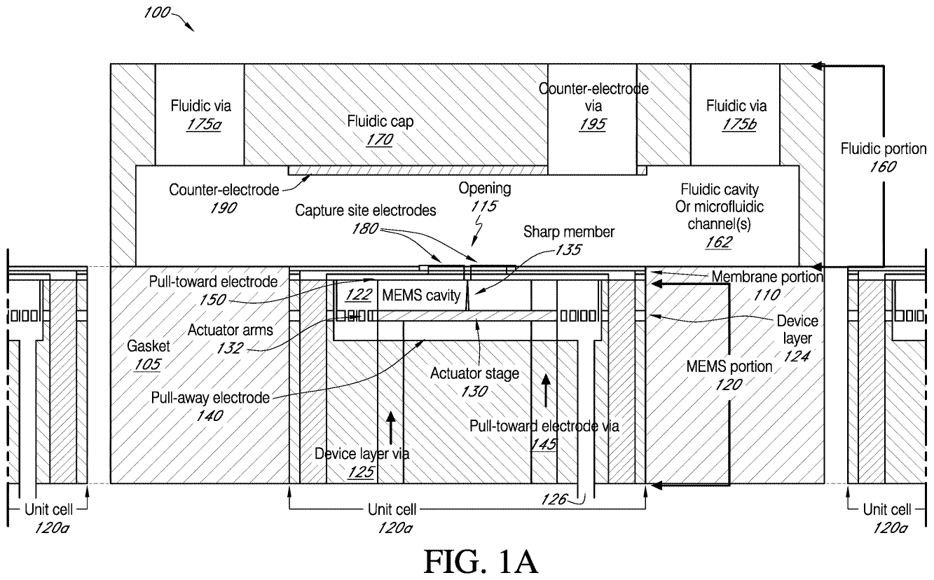

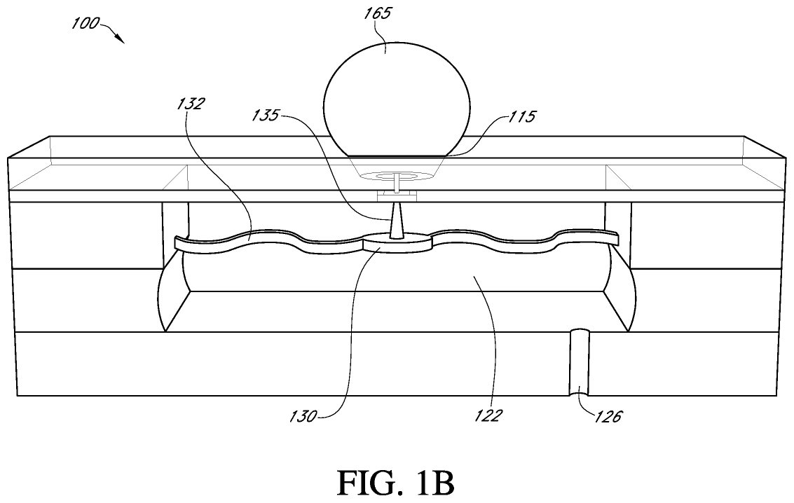

As described herein, the term “fluidic portion” is defined in FIG. 1A including a fluidic cavity (which may be subdivided into microfluidic channels), a fluidic cap, and a counter-electrode, which operates in conjunction with the capture site electrodes in the membrane portion to provide a DEP capture force.

As described herein, the term “functional layers” is a term that encompasses any permutation or combination of the following structural components bonded together and anchored to a surface. These structural components include chemical anchors (moieties interacting with an inorganic handle), spacers (groups intended to alter length of any surface layer), synthetic linkers (moieties intended to form irreversible bonds with subsequent structural components) and transporters (any chemical groups intended to reversibly bind payload molecules).

As described herein, the term “functionalization” refers to the formation of a thin film on a surface to alter its material properties or imbue new behaviors. Examples of this include the formations of thin films to modulate surface energy, or enable reversible binding of molecular payloads. Examples of thin films include monolayers, multilayer coatings, and polymer coatings.

As described herein, the term “inorganic handle” refers to the un-functionalized sharp member surface and is comprised of Au, for example, or SiOₓ, TiOₓ, AlOₓ, ITO, hydrogen-terminated silicon, SiNX, Pt, Ag, Ni, Cu, or other metals or metal oxides or nitrides, for example.

As described herein, the term “integrated control ASIC” refers to an application specific integrated circuit.

As described herein, the term “interconnects” refers to the in-plane routing within the 2D area of a functional layer.

As described herein, the term “interposer portion” refers to an electrical redistribution layer and/or an application-specific integrated circuit (ASIC) used to physically map the electrical signal I/O between different electrical contact layouts, or a combination of two or more of these configurations.

As described herein, the term “interrogate” refers to activities such as, for example, material sampling, physical probing, sensing, payload delivery, interaction, physical touching, capillary wicking, and/or insertion as discussed herein.

As described herein, the term “microchannels” refers to possible sub-sections of the fluidic cavity for the purpose of sectioning the LOC into regions. More broadly, a microchannel is any fluid-carrying cavity that has one or more dimensions at the micrometer scale.

As described herein, the term “micro-electro-mechanical-system (MEMS)” refers to micrometer-scale devices or systems comprising both mechanical and electrical elements.

As described herein, the term “mounted” refers to the socketing, a mechanism for coupling, bonding, applying elastic or polymer for thermal stress management, physical clamping, mechanical clamping for manual alignment of MEMS and fluidic portions. Mounting is reversible or irreversible.

As described herein, the term “membrane opening” refers to the opening in the membrane portion to allow for the sharp member to pass through the membrane portion from the MEMS cavity and into the fluidic cavity to interrogate the particle in the capture site.

As described herein, the term “particle” refers to an object or a group of objects that individually or together have a physical property. The particle has a composition that can include mixtures, including, but not limited to living cells, viruses, oil droplets, liposomes, micelles, reverse micelles, protein aggregates, polymers, surfactant assemblies or their combination. The particle can be an individual, or a plurality of, cell (or cells), virus (or viruses), bacterium or bacteria, or any organism(s), alive or dead. The particle can be free floating in a fluid, e.g., suspended in the fluid, can be adherent, can change shape, can merge, can split apart, etc.

As described herein, the term “pore” refers to an opening between two regions. The term “payload” includes any chemical compound, polymer, biological macromolecule, or combination.

As described herein, the term “signal” includes any electrical events, such as variations in voltage, current, frequency, phase, or duration that may comprise DC, AC, or a superposition of frequency components.

As described herein, the term “interference” refers to any electromagnetic disturbance that interrupts, obstructs, or otherwise degrades or limits the effective transmission or readout of a signal or signal component.

As described herein, the term “interrogation” refers to activities such as, for example, material sampling, physical probing, sensing, payload delivery, interaction, physical touching, capillary wicking, and/or insertion.

As described herein, the term “payloads” refers to anything being delivered into the particle including a chemical compound, polymer, biological molecule, nanoparticle, nanostructure, organic or inorganic molecule, or combination thereof.

As described herein, the term “pull-in” refers to the voltage applied to the actuator (pull-in voltage) and the position it reaches in the MEMS cavity (pull-in distance, at around ⅓ of the distance between the resting position of the actuator and the pull-toward or away electrodes, whichever is being used) where the MEMS “pull-in” configuration has been reached and the actuator transitions to the latching mode from the non-latching mode or vice versa.

As described herein, the term “via” generally refers to an electrical via unless fluidic via is explicitly stated and is generally a connection between functional layers of the LOC and is roughly perpendicular to the chip plane or is an out-of-plane connection.

As described herein, the term “wafer bonding” is a packaging technology on wafer-level for the fabrication of MEMS, nanoelectromechanical systems (NEMS), microelectronics and optoelectronics, ensuring a mechanically stable and hermetically sealed encapsulation. Wafer bonding, or its analogous die bonding and chip bonding, is irreversible and can also include their components, the control ASIC etc.

The foregoing information and the following detailed description with respect to the figures and illustrative examples of various aspects and implementations are disclosed in accordance with various embodiments as described herein.

FIG. 1A illustrates an embodiment of an integrated device 100 and FIG. 1B shows a schematic view of the integrated device of FIG. 1A; in accordance with various embodiments. As illustrated in the FIGS. 1A and 1B, the technology described with respect to the integrated device 100 includes various components integrated into a system that can be configured to capture nanoscale or microscale materials 165 (also referred to herein as “particle or particles 165”), and to manipulate or interrogate the materials at the nanoscale or cellular level. For such embodiments, the integrated device 100 can include, for example, but not limited to, a fluidics-based capture architecture and a MEMS-based sample interrogation architecture. In accordance with various embodiments, one or more methods of using the integrated device 100 are also provided.

The integrated device 100 illustrated in FIG. 1A includes a membrane portion 110, a MEMS portion 120 and a fluidic portion 160. As illustrated, the membrane portion 110 includes a membrane opening 115 and has a first side facing the MEMS portion 120 and a second side facing the fluidic portion 160. As illustrated in FIG. 1A, the membrane portion 110 is disposed between the MEMS portion 120 and the fluidic portion 160. The membrane opening 115 is an opening through which one or more nanoscale or micro scale materials 165 are captured, immobilized, or otherwise trapped, and which are then manipulated or interrogated at the nanoscale or cellular level. In other words, the membrane opening 115 provides access between the MEMS portion 120 and the fluidic portion 160.

In various implementations, the membrane opening 115 has a size (also referred to herein as a diameter if circular or a lateral dimension if any non-circular geometry) between about 0.1 nm to about 1 mm. In various implementations, the membrane opening 115 has a size between about 1 nm to about 100 nm, about 100 nm to about 1 μm, about 1 μm to about 10 μm, about 100 nm to about 10 μm, about 100 nm to about 25 μm, about 500 nm to about 5 μm, about 500 nm to about 10 μm, about 1 μm to about 100 μm, or about 1 μm to about 50 μm, inclusive of any size ranges therebetween.

In various embodiments, the MEMS portion 120 includes a MEMS cavity 122 and an actuator stage 130 that is disposed within the MEMS cavity 122. In various implementations, the MEMS portion 120 may include a unit cell 120a, which comprises the MEMS cavity 122 and the actuator stage 130 that is disposed within the MEMS cavity 122. In various implementations, the MEMS portion 120 may include a plurality of unit cells 120a, each of which comprises a MEMS cavity 122 and an actuator stage 130 that is disposed within the MEMS cavity 122. In various implementations, each of the unit cells 120 can be fluidically interconnected, for example, via a MEMS fluidic access via 126. In accordance with various embodiments, the fluid contained within the MEMS cavity 122 (e.g., used to provide fluidic interconnection) includes for example, but limited to, an aqueous fluid, an aqueous buffer, an organic solvent, a hydrophobic fluid, a gas, aqueous solution containing biological or chemical reagents, organic solvents, mineral oil, fluorinated oil, air, mixed gases for cell culture (e.g., 5% CO₂), inert gas, and the like.

In various embodiments, the actuator stage 130 includes a sharp member 135 disposed on the actuator stage 130 within the MEMS cavity 122. In various embodiments, the actuator stage 130 is a square shape plate or a rectangular shape plate. In various implementations, the actuator stage 130 has a shape comprising, for example, but not limited to, circular, ellipse, oval, square, rectangle, pentagon, or hexagon.

In various implementations, the actuator stage 130 has a lateral dimension between about 100 nm and about 10 cm. In various implementations, the actuator stage 130 has a lateral dimension between about 1 μm and about 1 cm, about 1 μm and about 1 mm, about 1 μm and about 800 μm, about 1 μm and about 600 μm, about 1 μm and about 500 μm, about 1 μm and about 400 μm, about 1 μm and about 300 μm, about 1 μm and about 200 μm, about 1 μm and about 100 μm, about 5 μm and about 500 μm, about 10 μm and about 500 μm, about 25 μm and about 500 μm, about 50 μm and about 500 μm, or about 100 μm and about 500 μm, inclusive of all dimensions therebetween.

In various implementations, the actuator stage 130 moves from the resting position for a distance between about 0.1 nm and about 10 mm. In various implementations, the actuator stage 130 moves from the resting position for a distance between about 1 nm and about 8 mm, about 1 nm and about 1 mm, about 10 nm and about 6 mm, about 100 nm and about 5 mm, about 1 μm and about 4 mm, about 1 μm and about 3 mm, about 1 μm and about 2 mm, about 1 μm and about 1 mm, about 1 μm and about 10 μm, about 100 nm and about 10 μm, about 10 μm and about 1 mm, about 25 μm and about 1 mm, about 50 μm and about 1 mm, or about 50 μm and about 2 mm, inclusive of any distance ranges therebetween.

In various implementations, the actuator stage 130 moves from the resting position for static displacement of a distance between about 1 nm and about 10 mm. In various implementations, the actuator stage 130 moves from the resting position for dynamic displacement of a distance between about 0.1 nm and about 100 μm. In various implementations, the actuator stage 130 is actuated in a static manner while concurrently having a superimposed vibrational dynamic movement. In various implementations, the dynamic movement of the actuator stage 130 is configured for sensing or measuring applications such as, for example, to facilitate modulation of payload release kinetics via agitation enhanced diffusion, or other kinetic actions as may be desirable.

In various implementations, the actuator stage 130 has a thickness between about 0.001 μm and about 10 mm. In various implementations, the actuator stage 130 has a thickness between about 0.01 μm and about 1 mm, about 0.01 μm and about 500 μm, about 0.01 μm and about 100 μm, about 0.01 μm and about 75 μm, about 0.01 μm and about 50 μm, about 0.01 μm and about 25 μm, about 0.01 μm and about 10 μm, about 0.1 μm and about 10 μm, about 0.1 μm and about 25 μm, about 0.1 μm and about 50 μm, about 0.1 μm and about 75 μm, about 0.1 μm and about 100 μm, about 0.1 μm and about 250 μm, about 0.1 μm and about 500 μm, or about 0.1 μm and about 1 mm, inclusive of any thickness ranges therebetween.

In various implementations, the actuator stage 130 has a first thickness and actuator arms 132 have a second thickness. In various implementations, the first thickness is the same as the second thickness. In various implementations, the first thickness differs from the second thickness.

In various implementations, the actuator stage 130 includes one of single crystal silicon, polycrystalline silicon, nanocrystalline silicon, amorphous silicon, or hydrogenated amorphous silicon. In various implementations, the actuator stage 130 can include a metal, a metallic alloy, a ceramic, a composite, or a polymer. In various implementations, the actuator stage 130 can include doped silicon, any allotrope of silicon, any inorganic glassy material or mixture, any inorganic polycrystalline material or mixture, any inorganic single crystalline material or mixture, any ceramic material comprising metal oxides, metalloid oxides, metal or metalloid nitrides, metal or metalloid oxides with nitrogen or other non-metalloid or metal elements, any doped combination of the above materials, any layered stack or structural combination of the above materials.

In various implementations, the actuator stage 130 is a single layer of material. In various implementations, the actuator stage 130 is a composite material having multiple layers. In various implementations, the actuator stage 130 can include an empty void layer, or one or more voids in the composite material.

In various implementations, the actuator stage 130 has a doping concentration between about 10¹⁰ atoms/cm³ and about 10²¹ atoms/cm³. In various implementations, the actuator stage 130 has a doping concentration between about 10¹⁰ atoms/cm³ and about 10²⁰ atoms/cm³, 10¹¹ atoms/cm³ and about 10²¹ atoms/cm³, or about 10¹¹ atoms/cm³ and about 10²⁰ atoms/cm³. In various implementations, the actuator stage 130 can be doped with a dopant from the list of boron, phosphorous, arsenic, indium, gallium, antimony, bismuth, lithium, germanium, nitrogen, and gold.

In various implementations, the actuator stage 130 has a resistivity value between about 10⁻⁷ Ω-cm and about 10⁶ Ω-cm. In various implementations, the actuator stage 130 has a resistivity value between about 10⁻⁶ Ω-cm and about 10⁵ Ω-cm, about 10⁻⁴ Ω-cm and about 10⁴ Ω-cm, or about 10⁻³ Ω-cm and about 10⁴ Ω-cm.

In various embodiments, a plurality of sharp members 135 are disposed on the actuator stage 130. In various implementations, the actuator stage 130 can accommodate a plurality of sharp members 135 up to about 2 sharp members, up to about 5 sharp members, up to about 10 sharp members, up to about 50 sharp members, up to about 100 sharp members, up to about 500 sharp members, up to about 1,000 sharp members, up to about 5,000 sharp members, up to about 10,000 sharp members, up to about 50,000 sharp members, up to about 100,000 sharp members, up to about 500,000 sharp members, up to about 1,000,000 sharp members, up to about 5,000,000 sharp members, up to about 10,000,000 sharp members, up to about 50,000,000 sharp members, up to about 100,000,000 sharp members, or up to about 500,000,000 sharp members, inclusive of any ranges of sharp members between any two numbers described above, or between two sharp members and any upper limit described above.

In various implementations, the sharp member 135 is a needle, a microneedle, nanoneedle, a nanotube, a pillar, a micropillar, a nanopillar, or any physical projection with an aspect ratio of height to diameter of about 2 to about 1,000,000. In various implementations, the sharp member 135 has an aspect ratio of height to diameter of about 1 to about 1,000,000, about 1 to about 500,000, about 1 to about 100,000, about 1 to about 50,000, about 1 to about 10,000, about 1 to about 5,000, about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 90, about 1 to about 80, about 1 to about 70, about 1 to about 60, about 1 to about 50, about 1 to about 40, about 1 to about 30, about 1 to about 20, about 1 to about 10, about 1 to about 9, about 1 to about 8, about 1 to about 7, about 1 to about 6, about 1 to about 5, about 1 to about 4, about 1 to about 3, about 1 to about 2, about 10 to about 1,000,000, about 10 to about 500,000, about 10 to about 100,000, about 10 to about 50,000, about 10 to about 10,000, about 10 to about 5,000, about 10 to about 1,000, about 10 to about 900, about 10 to about 800, about 10 to about 700, about 10 to about 600, about 10 to about 500, about 10 to about 400, about 10 to about 300, about 10 to about 200, about 10 to about 100, about 10 to about 90, about 10 to about 80, about 10 to about 70, about 10 to about 60, about 10 to about 50, about 10 to about 40, about 10 to about 30, about 10 to about 20, or about 5 to about 20, inclusive of any aspect ratio ranges therebetween.

In various implementations, the sharp member 135 includes insulating materials, such as silicon oxide or silicon nitride, or other metallic oxides, such as hafnium or aluminum oxide. In various implementations, the sharp member 135 is coated with a coating for chemical inertness and/or electrical insulation. In various implementations, the coating can be either deposited via a vapor phase or liquid phase deposition technique. In various implementations, the coating includes coating materials, such as, for example, inorganic insulators, such as oxides or nitrides, and various polymers that can be deposited or polymerized in-situ. In various implementations, the coating includes for example, a vapor phase deposited polymer coating, such as Parylene. In various implementations, the coating can also include materials that modify the surface properties or provide reactive groups for attaching molecules. These include the organosilanes, such as alkyl silanes, dichloro or trichloro silanes or trimethoxy silanes, fluorinated alkyl silanes, and silanes with reactive groups designed to change the surface properties such as aminosilanes, and methoxy or ethoxy silanes. Another category of coatings that can be applied in a liquid phase are those used in electrophoresis, such as polyacrylamides, polydimethylacrylamide, agarose, and other polysaccharides, such as guaran or locust bean gum. Yet another category of surface coatings can include surfactant molecules, in particular nonionic surfactants, such as Pluronic, a triblock copolymer with polypropylene oxide and polyethylene oxide segments. In various implementations, the coatings include multiple layers of coatings to help achieve various goals, such as an electrically insulating layer followed by a layer to promote molecular binding. In various implementations, the coating on the sharp member 135 is to help facilitate binding of certain chemicals to be inserted into a cell.

In various implementations, the coating can be any material suitable for providing chemical inertness, electrical insulation, and/or fluid wettability (e.g., for contact angle control). In various implementations, the coating can include, for example, a hydrophilic coating that includes a range of material classes, including for example, any small molecule, proteins, peptides, peptoids, polymers, or inorganic material, such as silicon, alumina, a ceramic, gold, silicon oxide, a metal, a polymer, a layered stack of these materials, of/or any combination either physically absorbed and/or deposited, or chemically bound covalently or non-covalently. In various implementations, the coating can include, for example, a hydrophobic coating that has a contact angle between about 95° and about 165°. In various implementations, the hydrophobic coating has a contact angle between about 95° and about 165°, between about 100° and about 165°, about 105° and about 165°, about 110° and about 165°, about 120° and about 165°, about 95° and about 150°, about 95° and about 140°, or about 95° and about 130°, inclusive of any contact angle ranges therebetween.

In various implementations, the hydrophilic coating has a contact angle between about 20° and about 80°. In various implementations, the hydrophilic coating has a contact angle between about 25° and about 80°, about 30° and about 80°, about 35° and about 80°, about 40° and about 80°, about 20° and about 70°, about 20° and about 60°, or about 20° and about 50°, inclusive of any contact angle ranges therebetween.

In various implementations, the sharp member 135 has a combination of patterned hydrophilic and hydrophobic coatings. The hydrophobic coating can include a variety of classes such as azides, organosilanes, hydrocarbons, or fluorocarbons, or organic molecules covalently bound or non-covalently bound. In various implementations, the coating on the sharp member 135 is to help facilitate binding of certain chemicals to be inserted into a cell.

In various implementations, the sharp member 135 has a length between about 50 nm and about 1 mm. In various implementations, the sharp member 135 has a length between about 50 nm and about 50 μm, between about 100 nm and about 25 μm, between about 100 nm and about 10 μm, between about 100 nm and about 5 μm, between about 100 nm and about 2 μm, between about 200 nm and about 25 μm, between about 200 nm and about 10 μm, between about 200 nm and about 5 μm, between about 200 nm and about 2 μm, between about 300 nm and about 25 μm, between about 300 nm and about 10 μm, between about 300 nm and about 5 μm, between about 300 nm and about 2 μm, between about 1 μm and about 1 mm, about 1 μm and about 500 μm, about 1 μm and about 250 μm, about 1 μm and about 100 μm, about 1 μm and about 75 μm, about 1 μm and about 50 μm, about 1 μm and about 20 μm, about 2 μm and about 20 μm, about 2 μm and about 50 μm, about 2 μm and about 75 μm, about 2 μm and about 100 μm, about 2 μm and about 250 μm, about 2 μm and about 500 μm, or about 2 μm and about 1 mm, inclusive of any length ranges therebetween.

In various implementations, the sharp member 135 can have a distal (or base) portion attached substantially perpendicular to the actuator stage 130. In various implementations, the sharp member 135 can have a tip (also referred to herein as “proximal” portion) of the sharp member aligned or substantially aligned with the membrane opening 115.

In various implementations, in order to utilize the sharp member 135 for gene therapy, for example, in in-vitro cellular transformation, microscale cells are typically collected, selected, and held in place (otherwise suspended in place) prior to insertion. As such, the dimensions of the sharp member 135 is on the scale of cellular dimensions, i.e., in the nanometer range. In various embodiments, the dimensions of the sharp member 135 is one order or two orders of magnitude smaller than cellular dimensions. For the desired precision and controllability, the sharp member 135 is sharp enough to penetrate a cellular membrane and a nuclear membrane with minimal force and minimal disruption to the cell outside the penetration point. In various embodiments, the sharp member 135 is capable of being actuated over a sufficient distance, typically in the order of the cell dimensions, i.e., from about 2 μm to about 20 μm, and preferably from about 4 μm to about 8 μm. In various embodiments, the sharp member 135 has an individual actuation mechanism that enables accurate movement relative to the cell that is being held in place. In various embodiments, the actuation mechanism for each of the sharp member 135 is compact so that the total area of the sharp member 135 and its actuation mechanism is small.

In various embodiments, the sharp member 135 includes silicon. In various embodiments, the sharp member 135 includes a sharp tip. In various embodiments, the sharp member 135 has a hollow inner portion and a coated tip. In various embodiments, the sharp member 135 has a coating disposed on its sharp tip. In various embodiments, the sharp member 135 has a hollow inner portion and a coating disposed on its tip. In various embodiments, the sharp member 135 is coated with one or more materials configured for conjugation of a polynucleotide to the sharp member 135. In various embodiments, at least a portion of the sharp member 135 is coated with a plurality of gold atoms. In various embodiments, the sharp member 135 coated with gold atoms is capable of being attached to one or more biological molecules. The capability to be attached one or more biological molecules is due to unique properties of the gold coated sharp member 135, which includes a unique surface, chemical inertness, high electron density, and strong optical absorption.

In various embodiments, the sharp member 135 can include at least one of a Langmuir-Bodgett film, a functionalized glass, germanium, PTFE, polystyrene, gallium arsenide, silver, membrane, nylon, PVP, silicon oxide, metal oxide, or ceramics. In various embodiments, the sharp member 135 is capable of having functional groups such as, for example, amino, carboxyl, Diels-Alder reactants, thiol or hydroxyl incorporated on its surface. Such materials allow the attachment of the nucleic acids and their interaction with target molecules without hindrance from the sharp member 135.

In various embodiments, the sharp member 135 can be conjugated to a polynucleotide via a covalent bond, a thiol (—SH) modifier, avidin/biotin coupling chemistry, or a mediating linker molecule from one of polyethylene glycol (PEG) or polyethyleneimine (PEI). In various embodiments, the sharp member 135 is capable of carrying a payload including, for example, nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs, exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs.

In various embodiments, the sharp member 135 is capable of carrying a genetic material capable of stably integrating into a cell's genome from a list of gRNA comprising crRNA or a tracrRNA capable of complexing with a Cas protein or plasmid expressing Cas protein, TALENs or zinc finger nucleases.

In various embodiments, the actuator stage 130 suspended within the MEMS cavity 122 and supported by two or more actuator arms 132 attached to walls of the MEMS cavity 122. In various embodiments, the two or more actuator arms 132 can include a serpentine pattern and/or be made of a conductive material.

In various implementations, the two or more actuator arms 132 include one of single crystal silicon, polycrystalline silicon, nanocrystalline silicon, amorphous silicon, or hydrogenated amorphous silicon. In various implementations, the two or more actuator arms 132 can include a metal, a metallic alloy, a ceramic, a composite, or a polymer. In various implementations, the two or more actuator arms 132 can include doped silicon, any allotrope of silicon, any inorganic glassy material or mixture, any inorganic polycrystalline material or mixture, any inorganic single crystalline material or mixture, any ceramic material comprising metal oxides, metalloid oxides, metal or metalloid nitrides, metal or metalloid oxides with nitrogen or other non-metalloid or metal elements, any doped combination of the above materials, any layered stack or structural combination of the above materials.

In various implementations, the two or more actuator arms 132 include a single layer of materials. In various implementations, the two or more actuator arms 132 include a composite material having multiple layers. In various implementations, the two or more actuator arms 132 can include an empty void layer, or one or more voids in the composite material.

In various implementations, the two or more actuator arms 132 have a thickness between about 0.001 μm and about 10 mm. In various implementations, the two or more actuator arms 132 have a thickness between about 0.01 μm and about 1 mm, about 0.01 μm and about 500 μm, about 0.01 μm and about 100 μm, about 0.01 μm and about 75 μm, about 0.01 μm and about 50 μm, about 0.01 μm and about 25 μm, about 0.01 μm and about 10 μm, about 0.1 μm and about 10 μm, about 0.1 μm and about 25 μm, about 0.1 μm and about 50 μm, about 0.1 μm and about 75 μm, about 0.1 μm and about 100 μm, about 0.1 μm and about 250 μm, about 0.1 μm and about 500 μm, or about 0.1 μm and about 1 mm, inclusive of any thickness ranges therebetween.

In various implementations, the two or more actuator arms 132 have a doping concentration between about 10¹⁰ atoms/cm³ and about 10²¹ atoms/cm³. In various implementations, the two or more actuator arms 132 have a doping concentration between about 10¹⁰ atoms/cm³ and about 10²⁰ atoms/cm³, 10¹¹ atoms/cm³ and about 10²¹ atoms/cm³, or about 10¹¹ atoms/cm³ and about 10²⁰ atoms/cm³. In various implementations, the two or more actuator arms 132 can be doped with a dopant from the list of boron, phosphorous, arsenic, indium, gallium, antimony, bismuth, lithium, germanium, nitrogen, and gold.

In various implementations, the two or more actuator arms 132 are configured to be flexible, for example, for bending without plastic deformation, material fatigue, and fracture in the two or more actuator arms 132 after repeated movements. In various implementations, the two or more actuator arms 132 are configured to support mechanical vibrations of the actuator stage 130. In various implementations, the two or more actuator arms 132 are fabricated from the same material layer as the actuator stage 130. In various implementations, the two or more actuator arms 132 have the same electrical properties, for example, electrical resistance and impedance as the actuator stage 130.

In various implementations, the two or more actuator arms 132 have a resistivity value between about 10⁻⁷ Ω-cm and about 10⁶ Ω-cm. In various implementations, the two or more actuator arms 132 have a resistivity value between about 10⁻⁶ a-cm and about10⁵ Ω-cm, about 10⁻⁴ Ω-cm and about 10⁴ Ω-cm, or about 10⁻³ Ω-cm and about 10⁴ Ω-cm.

In various embodiments, the actuator stage 130 is suspended a predetermined distance away from the (first) surface of the membrane portion 110. When actuated or in operation, the actuator stage 130 can be configured to move relative to the surface of the membrane portion so as to move the tip of the sharp member 135 through the membrane opening 115. In various embodiments, the tip of the sharp member 135 moves across the membrane opening 115 and into at least a portion of the fluidic cavity 160.

As illustrated in FIG. 1A, the sharp member 135 is disposed on a first surface (e.g., top surface) of the actuator stage 135. In various embodiments, the actuator stage 135 can act as an electrode and the MEMS cavity 122 can include one or more pull-away electrodes 140 disposed on the surface below the actuator stage 130, e.g., the bottom surface of the MEMS cavity 122 as shown in FIG. 1A. In various embodiments, the MEMS cavity 122 may include one or more of its walls as part of the pull-away electrodes 140. As such, the one or more pull-away electrodes 140 disposed on the surface below the actuator stage 130 is facing a second surface (e.g., bottom surface) of the actuator stage 130. In accordance with various embodiments, the actuator stage 130 and the one or more pull-away electrodes 140 can be configured as components of a parallel plate electrostatic actuation device architecture. In various embodiments, the MEMS portion 120 includes a device layer 124 and a device layer via 125 that is configured to electrically connect to the actuator stage 130.

In various implementations, the actuator stage 130 is suspended from the one or more pull-away electrodes 140 at a separation distance between about 10 nm and about 100 nm, about 10 nm and about 500 nm, about 10 nm and about 1 μm, about 100 nm and about 1 μm, about 1 μm and about 1 mm, about 1 μm and about 500 μm, about 1 μm and about 250 μm, about 1 μm and about 100 μm, about 1 μm and about 75 μm, about 1 μm and about 50 μm, about 2 μm and about 50 μm, about 2 μm and about 75 μm, about 2 μm and about 100 μm, about 2 μm and about 250 μm, about 2 μm and about 500 μm, or about 2 μm and about 1 mm, inclusive of any separation distance ranges therebetween.

In various implementations, the one or more pull-away electrodes 140 have a thickness between about 0.1 nm and about 10 mm. In various implementations, the one or more pull-away electrodes 140 have a thickness between about 0.001 μm and about 1 mm, about 0.01 μm and about 500 μm, about 0.01 μm and about 100 μm, about 0.01 μm and about 75 μm, about 0.01 μm and about 50 μm, about 0.01 μm and about 25 μm, about 0.01 μm and about 10 μm, about 0.1 μm and about 10 μm, about 0.1 μm and about 25 μm, about 0.1 μm and about 50 μm, about 0.1 μm and about 75 μm, about 0.1 μm and about 100 μm, about 0.1 μm and about 250 μm, about 0.1 μm and about 500 μm, or about 0.1 μm and about 1 mm, inclusive of any thickness ranges therebetween.

In various implementations, the one or more pull-away electrodes 140 include one of single crystal silicon, polycrystalline silicon, nanocrystalline silicon, amorphous silicon, or hydrogenated amorphous silicon. In various implementations, the one or more pull-away electrodes 140 can include a metal, a metallic alloy, a ceramic, a composite, or a polymer. In various implementations, the counter-electrode can include doped silicon, any allotrope of silicon, any inorganic glassy material or mixture, any inorganic polycrystalline material or mixture, any inorganic single crystalline material or mixture, any ceramic material comprising metal oxides, metalloid oxides, metal or metalloid nitrides, metal or metalloid oxides with nitrogen or other non-metalloid or metal elements, any doped combination of the above materials, any layered stack or structural combination of the above materials. In various implementations, the one or more pull-away electrodes 140 can include indium-tin oxide (ITO), titanium nitride (TiN), a metal film, a doped semiconducting film, an inorganic semiconductor, a composite, an organic conducting film, any carbon allotrope including various types of graphene, a graphene oxide, mismatched graphene, and any combination thereof.

In various implementations, the one or more pull-away electrodes 140 include a single layer of materials. In various implementations, the one or more pull-away electrodes 140 include a composite material having multiple layers. In various implementations, the one or more pull-away electrodes 140 can include an empty void layer, or one or more voids in the composite material.

In various implementations, the one or more pull-away electrodes 140 have a doping concentration between about 10¹⁰ atoms/cm³ and about 10²¹ atoms/cm³. In various implementations, the one or more pull-away electrodes 140 have a doping concentration between about 10¹⁰ atoms/cm³ and about 10²⁰ atoms/cm³, 10¹¹ atoms/cm³ and about 10²¹ atoms/cm³, or about 10¹¹ atoms/cm³ and about 10²⁰ atoms/cm³. In various implementations, the one or more pull-away electrodes 140 can be doped with a dopant from the list of boron, phosphorous, arsenic, indium, gallium, antimony, bismuth, lithium, germanium, nitrogen, and gold.

In various implementations, the one or more pull-away electrodes 140 have a resistivity value between about 10⁻⁷ Ω-cm and about 10⁶ Ω-cm. In various implementations, the one or more pull-away electrodes 140 have a resistivity value between about 10⁻⁶ Ω-cm and about 10⁵ Ω-cm, about 10⁻⁴ Ω-cm and about 10⁴ Ω-cm, or about 10⁻³ Ω-cm and about 10⁴ a-cm.

As illustrated in FIG. 1A, the first side (e.g., downward facing side) of the membrane portion 110 includes one or more pull-toward electrodes 150 disposed on a first surface (e.g., downward facing surface) of the membrane portion 110. In various implementations, the one or more pull-toward electrodes 150 are disposed adjacent to the membrane opening 115 and/or at least partially surrounding the membrane opening 115. In various embodiments, the one or more pull-toward electrodes 150 are disposed partially bracketing the membrane opening 115. In various embodiments, the one or more pull-toward electrodes 150 at least partially surrounds the membrane opening 115 in at least one dimension. In various embodiments, the one or more pull-toward electrodes 150 at least substantially encloses the membrane opening 115 in at least one dimension. In various embodiments, the one or more pull-toward electrodes 150 are electrically connected to form a circular pull-toward electrode. In various embodiments, the one or more pull-toward electrodes 150 are electrically connected to form an electrode in the shape of a square, rectangular, triangular, pentagonal, hexagonal, or oval, etc. In various embodiments, the MEMS portion 120 includes a pull-toward electrode via 145 (or multiple vias 145) that is configured to provide an electrical connection between the one or more pull-toward electrodes 150 and a power source (not shown). In accordance with various embodiments, the actuator stage 130 and the one or more pull-toward electrodes 150 can be configured as components of a parallel plate electrostatic actuation device architecture.

In various implementations, the actuator stage 130 is suspended from the one or more pull-toward electrodes 150 at a separation distance between about 10 nm and about 100 nm, about 10 nm and about 500 nm, about 10 nm and about 1 μm, about 100 nm and about 1 μm, about 1 μm and about 1 mm, about 1 μm and about 500 μm, about 1 μm and about 250 μm, about 1 μm and about 100 μm, about 1 μm and about 75 μm, about 1 μm and about 50 μm, about 2 μm and about 50 μm, about 2 μm and about 75 μm, about 2 μm and about 100 μm, about 2 μm and about 250 μm, about 2 μm and about 500 μm, or about 2 μm and about 1 mm, inclusive of any separation distance ranges therebetween.

In various implementations, the one or more pull-toward electrodes 150 have a thickness between about 0.1 nm and about 10 mm. In various implementations, the one or more pull-toward electrodes 150 have a thickness between about 0.001 μm and about 1 mm, about 0.01 μm and about 500 μm, about 0.01 μm and about 100 μm, about 0.01 μm and about 75 μm, about 0.01 μm and about 50 μm, about 0.01 μm and about 25 μm, about 0.01 μm and about 10 μm, about 0.1 μm and about 10 μm, about 0.1 μm and about 25 μm, about 0.1 μm and about 50 μm, about 0.1 μm and about 75 μm, about 0.1 μm and about 100 μm, about 0.1 μm and about 250 μm, about 0.1 μm and about 500 μm, or about 0.1 μm and about 1 mm, inclusive of any thickness ranges therebetween.

In various implementations, the one or more pull-toward electrodes 150 include one of single crystal silicon, polycrystalline silicon, nanocrystalline silicon, amorphous silicon, or hydrogenated amorphous silicon. In various implementations, the one or more pull-toward electrodes 150 can include a metal, a metallic alloy, a ceramic, a composite, or a polymer. In various implementations, the one or more pull-toward electrodes 150 can include doped silicon, any allotrope of silicon, any inorganic glassy material or mixture, any inorganic polycrystalline material or mixture, any inorganic single crystalline material or mixture, any ceramic material comprising metal oxides, metalloid oxides, metal or metalloid nitrides, metal or metalloid oxides with nitrogen or other non-metalloid or metal elements, any doped combination of the above materials, any layered stack or structural combination of the above materials. In various implementations, the one or more pull-toward electrodes 150 can include indium-tin oxide (ITO), titanium nitride (TiN), a metal film, a doped semiconducting film, an inorganic semiconductor, a composite, an organic conducting film, any carbon allotrope including various types of graphene, a graphene oxide, mismatched graphene, and any combination thereof.

In various implementations, the one or more pull-toward electrodes 150 include a single layer of materials. In various implementations, the one or more pull-toward electrodes 150 include a composite material having multiple layers. In various implementations, the one or more pull-toward electrodes 150 can include an empty void layer, or one or more voids in the composite material.

In various implementations, the one or more pull-toward electrodes 150 can have a doping concentration between about 10¹⁰ atoms/cm³ and about 10²¹ atoms/cm³. In various implementations, the one or more pull-toward electrodes 150 have a doping concentration between about 10¹⁰ atoms/cm³ and about 10²⁰ atoms/cm³, 10¹¹ atoms/cm³ and about 10²¹ atoms/cm³, or about 10¹¹ atoms/cm³ and about 10²⁰ atoms/cm³. In various implementations, the one or more pull-toward electrodes 150 can be doped with a dopant from the list of boron, phosphorous, arsenic, indium, gallium, antimony, bismuth, lithium, germanium, nitrogen, and gold.

In various implementations, the one or more pull-toward electrodes 150 have a resistivity value between about 10⁻⁷ Ω-cm and about 10⁶ Ω-cm. In various implementations, the one or more pull-toward electrodes 150 have a resistivity value between about 10⁻⁶ Ω-cm and about 10⁵ Ω-cm, about 10⁻⁴ Ω-cm and about 10⁴ Ω-cm, or about 10⁻³ Ω-cm and about 10⁴ Ω-cm.

In various embodiments, the second side (e.g., top side) of the membrane portion 110 includes one or more capture-site electrodes 180 disposed on a second surface (e.g., top surface) of the membrane portion 110. In various embodiments, the one or more capture-site electrodes 180 are disposed adjacent to the membrane opening 115. In various embodiments, at least a portion of the one or more capture-site electrodes 180 are partially or fully embedded in the second side of the membrane portion 110. In various embodiments, the one or more capture-site electrodes 180 include a circular capture-site electrode geometry or an annular capture-site electrode geometry. In various embodiments, the one or more capture-site electrodes 180 include a pair of bi-polar electrodes that are disposed across the membrane opening 115.

In various embodiments, the fluidic portion 160 is disposed on the second side (e.g., above the membrane portion 110) of the membrane portion 110. As illustrated in FIG. 1A, the fluidic portion 160 includes a fluidic cap 170 that forms a fluidic cavity (e.g., a channel, such as a microfluidic channel) 162 for flowing a fluid medium (not shown) within the fluidic portion 160. In various embodiments, the fluidic medium includes nanoscale or microscale materials 165 in the fluidic medium. In various embodiments, the fluidic cap 170 includes a fluidic via 175a as a fluidic inlet and a fluidic via 175b as a fluidic outlet (collectively referred to herein as “fluidic via 175”) for fluidic input and output within the fluidic portion 160. In various embodiments, the fluid medium flows from the fluidic inlet to the fluidic outlet at a predetermined flow rate. In various embodiments, the fluidic portion 160 can include multiple fluidic inlets and multiple fluidic outlets.

In various embodiments, the fluidic cap 170 includes an optically transparent material for specific wavelengths. In various embodiments, the fluidic cap 170 includes a transparent material like SiOₓ or an optically transparent thermoplastic. In various embodiments, the fluidic cap 170 includes optically opaque materials, such as for example, but not limited to, silicon or gold. In various embodiments, the fluidic cap 170 includes a soft material, such as an elastomer.

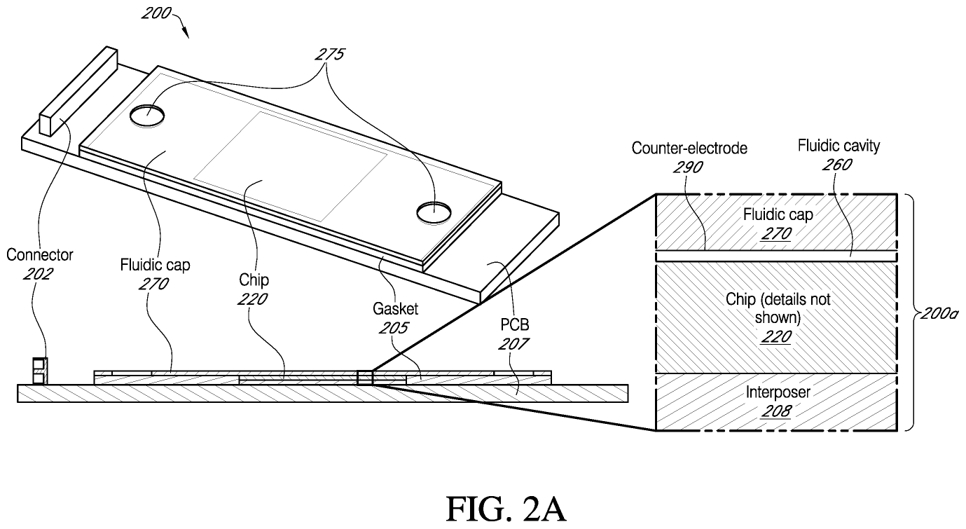

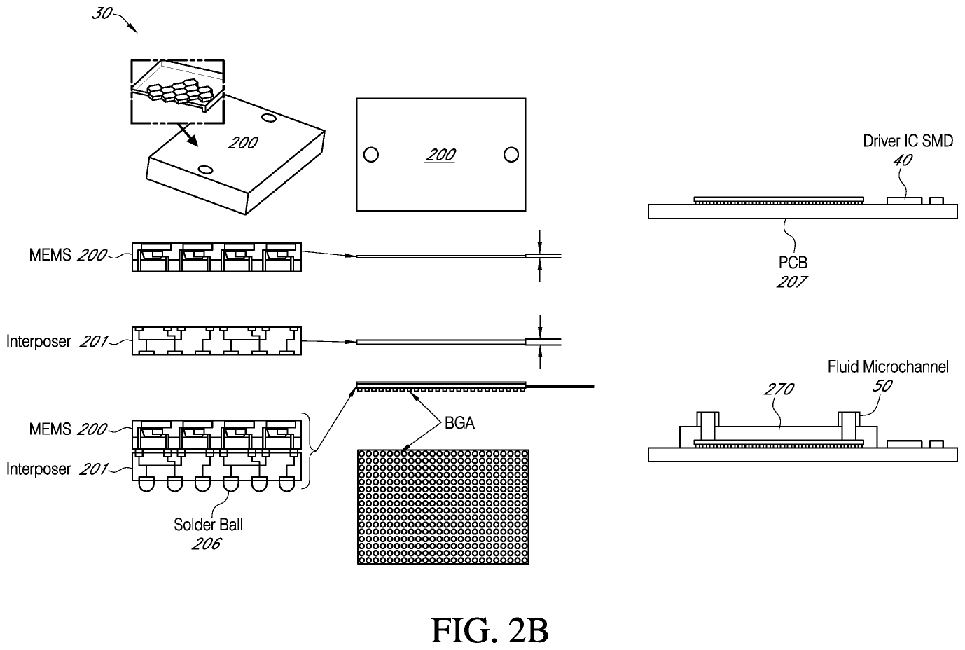

In various embodiments, the fluidic cavity 162 can be divided into spatially discrete regions (e.g., microchannels) by dividing walls. In various embodiments, the fluidic cap 170 may be bonded directly to the membrane portion 110. In various embodiments, the fluidic cap 170 may be mounted (irreversibly or reversibly) to a surrounding gasket, such as gasket 205 of FIG. 2A, which is in-plane with the chip 220 of FIG. 2A, and conforms to its edges. This approach serves the purpose of sealing the fluidic cavity in a way which avoids occupying chip surface area in order to make available the maximum possible number of capture sites, for example. The fluidic portion also may contain a counter-electrode located on the fluidic cap across the fluidic cavity from the membrane portion. In some embodiments, this counter-electrode works in concert with the capture site electrodes on the membrane portion in order to provide an electric field for dielectrophoretic capture of particles at the capture sites.

In various embodiments, the fluidic portion 160 includes one or more counter-electrodes 190 disposed on a fluidic cavity surface (e.g., at bottom surface of the fluidic cap 170) across the fluidic cavity 162 from the membrane opening 115. In various embodiments, the fluidic portion 160 includes a counter-electrode via 195 (or multiple vias 195) that is configured to provide an electrical connection between the one or more counter-electrodes 190 and a power source (not shown). In various embodiments, the one or more counter-electrodes 190 includes an optically transparent conductive film, such as indium tin oxide (ITO). In various embodiments, the one or more counter-electrodes 190 includes an optically opaque conductive film, such as gold or silver. In various embodiments, the one or more counter-electrodes 190 includes polymer glass (organic and inorganic). In various implementations, the one or more counter-electrodes 190 and the one or more capture-site electrodes 180 are configured as one or more electrode pairs configured to capture nanoscale or microscale materials (e.g., one or more particles) 165 in the fluid medium. In various implementations, the integrated device 100 can be electrically coupled to a power source configured for providing power to the integrated device 100.

In accordance with various embodiments, the nanoscale or microscale materials (one or more particles) 165 can be captured using dielectrophoretic (DEP) force. The DEP approach can be particularly useful for local manipulation of neutral particles or biological molecules in a fluidic and non-linear electric field environment, e.g., microfluidics applications. In particular, DEP-based trapping, capturing, or immobilization of nanoscale or microscale materials, such as, biological objects, single cells or groups of cells in proximity to a compartment (or cavity) for local manipulation of the molecules or cells. The DEP force is nominally expressed as follows:

F_{DEP}=π∈ₘr³Re(f_{CM})∇|E|²

where r is the radius of the particle, ∈ₘ is the permittivity of the fluid, E is the electric field, and f_{CM} is the Clausius-Mossotti factor, a complex value that depends on the difference in permittivity between the fluid and the particle, and which determines if the DEP force will be positive or negative.

In various implementations, the fluidic cavity 162 can be filled with the fluidic medium including, for example, an aqueous fluid, an aqueous buffer, an organic solvent, a hydrophobic fluid, or a gas. In various implementations, the fluidic cavity 162 can contain a fluid within the fluidic cavity 162 that is immiscible with a fluid outside the fluidic cavity 162, such as the MEMS cavity 122. In various implementations, the fluidic cavity 162 can contain a non-aqueous fluid or microelectronics. Suitable applications that the DEP-based technique can be applied to include interrogation of discrete biologics, e.g., interrogation or probing of cells, living cells, viruses, oil droplets, liposomes, micelles, reverse micelles, protein aggregates, polymers, surfactant assemblies or their combination, etc.

The technology disclosed herein relates to coupling aqueous microfluidic environments with structures that can be in non-aqueous environments, e.g., electronics that can be in a non-conductive fluid or processes which can use hydrophobic solvents. The disclosed technology can offer local manipulation of isolated particles in a fluidic environment at scale, while allowing access from a compartment that contains sensitive MEMS components or electronics, such as those that can be included in the MEMS cavity 122 of the MEMS portion 122, is disclosed. This can be done by coupling MEMS processes (in MEMS portion 120) with microfluidic processes (in fluidic portion 160) to allow for high-throughput processing and interrogation of particles (the term particle or particles may refer to “biological object, objects or cells” and non-biological objects). In particular, the technology described herein relates to a high-throughput, DEP-based particle immobilization (trapping) apparatus that pins/immobilizes one or more particles in a fluid, which flows adjacent to the membrane portion 110 that separates the fluid from the MEMS cavity 122, wherein the MEMS cavity 122 contains electronic components, including the sharp member 135, the actuator stage 130, and various components. In various embodiments, the DEP force is applied perpendicular to the membrane opening 115 in the fluidic portion 160.

In various embodiments, the fluidic portion 160 may be directly bonded to the membrane portion 110 and/or MEMS portion 120. In some embodiments, the fluidic portion 160 may not be bonded directly to the membrane portion 110 and/or MEMS portion 120, and it may instead be incorporated during the packaging process as described below with respect to FIG. 2. Similarly, the nanoscale or microscale materials 165 can be captured or trapped in close proximity to the MEMS portion 120, such that the thickness of the membrane portion 110 between the MEMS portion 120 and fluidic portion 160 is below a threshold permitting interaction between the two regions. In accordance with various embodiments, the membrane portion 110 serves a variety of purposes depending on application, and typically serves the function of separating the chemical and electrical environments within each of the MEMS cavity 122 and the fluidic portion 160. In various embodiments, the MEMS cavity 122 is separated chemically and electrically from the fluidic cavity 162, as described above.

In accordance with various embodiments, specific applications of the integrated device 100 further include one or more chemical coatings or one or more functional layers (monolayers or thin films, un-patterned or patterned onto a surface) on various exposed surfaces and materials throughout the entire MEMS cavity 122, the sharp member 135 and its surface, either or both of surfaces of the membrane portion 110, including inside and/or around the membrane opening 115, including the surface of the membrane portion 110 exposed to the fluidic cavity 162, and/or the surface of the fluidic cap 170 or microchannels 162. In various embodiments, these chemical coatings may be adsorbed to the applicable surface by chemisorption (e.g., covalent or ionic bonding) or physisorption (e.g., van der Waals force), and generally serve the purpose of modulating the surface energy in order to prevent buildup of material deposits, promote or reduce non-specific adhesion in particular areas, or to control wetting at specific boundaries.

In various embodiments, the chemical configurations and methods for specific applications in which payloads (e.g., any chemical compound, polymer, biological molecule, nanoparticle, micro- or nanostructure, organic or inorganic molecule, or combination) are to be delivered into captured, trapped, or immobilized microscale or nanoscale materials 165, such as discrete biological systems (e.g., cells) and abiotic systems (e.g., lipid vesicles, emulsions, or other multiphase systems). In various embodiments, these aptamer-based coatings can be used to functionalize the sharp member 135 allowing for the delivery of a wide range of payloads into captured cells, for example. In accordance with various embodiments, the integrated device 100 can be operated to capture a plurality of cells and deliver payloads that have been reversibly chemisorbed onto the sharp member 135 residing within the MEMS cavity 122.

In various embodiments, the un-functionalized surface of the sharp member 135 can be referred to as an “inorganic handle”. In accordance with various embodiments, the aptamer-based payload delivery system and methods can utilize organic molecules attached to an inorganic handle, for instance the surface of the sharp member 135. In various embodiments, gold is coated on the sharp member 135 as an inorganic handle to which a thiol bond is exploited as a means of affixing organic molecules of choice to the inorganic handle. In various embodiments, SiOₓ, TiOₓ, AlOₓ, ITO, hydrogen-terminated silicon, SiNₓ, Pt, Ag, Ni, Cu, or other metals or metal oxides or nitrides can also be utilized as an inorganic handle material. In some embodiments, the SiOₓ inorganic handle can be functionalized using organosilanes.

In some embodiments, using SiOₓ as an inorganic handle, a payload may be chemisorbed in a layered fashion on a functionalized surface for specific applications within different embodiments. In some embodiments, chemisorption of payloads to the sharp member 135 may occur spontaneously by mechanisms including, but not limited to inorganic chelation, non-covalent complementary binding (i.e., nucleic acids), or the formation of reversible covalent bonds. Other embodiments may include mechanisms of payload chemisorption (such as voltage induced coulombic attraction). Subsequent payload release can occur either through displacement by specific cellular metabolites, modulation of payload or surface oxidation state, or other means of desorption.

Referring now to FIG. 2A, an embodiment of an integrated device 200 packaged as a lab-on-chip (LOC) system, with a zoomed-in portion 200a illustrating various layering in the LOC system, in accordance with various embodiments. The integrated device 200 is substantially similar or identical to the integrated device 100, and therefore the details of each of its components will not be described in further detail unless they are different.

As illustrated in FIG. 2A, the integrated device 200 is bonded, mounted, or attached to a printed circuit board (PCB) 207, which includes connector 202 for providing electrical connections between the integrated device 200 and an external source or device (not shown). The integrated device 200 can be considered a core of the LOC system (and simple referred to herein as the “LOC”) that is packaged as an integrated package. As shown in FIG. 2A, the integrated device 200 includes fluidic vias 275, fluidic cap 270, and chip (e.g., MEMS portion) 220. The zoomed-in portion 200a illustrates one or more counter-electrodes 290 at a surface of the fluidic cap 270 facing a fluidic cavity 260. In various embodiments, the fluidic vias 275, the fluidic cap 270, the fluidic cavity 260, and the chip 220 are substantially similar or identical to their counterparts the fluidic vias 175, the fluidic cap 170, the fluidic cavity 160, and the MEMS portion 120 of the integrated device 100 of FIGS. 1A and 1B.

As shown in FIG. 2A, an interposer 208 is disposed between the integrated device 200 (e.g., the LOC) and the PCB 207. In various embodiments, the interposer 208 can include one or both of an electrical redistribution layer or an application-specific integrated circuit (ASIC). In various embodiments, the ASIC is configured to physically map electrical signal inputs and outputs between different electrical contact layouts, or a combination thereof.

In accordance with various implementations, the actuator stage, such as the actuator stage 130, is actuated via an applied electrostatic force. In various embodiments, the integrated device 200 includes one or more electrodes for providing the electrostatic force used for actuating the actuator stage. According to various implementations, the actuator stage 130 can be actuated via any suitable actuation approach, including electrostatic actuation, piezoelectric actuation, actuation using magnetic forces by attaching magnets to the actuators, or actuation based on temperature differences, such as by employing bimetallic elements or those based on shape memory alloys. The temperatures needed for actuation can be generated by fabricated resistive heaters in selected locations of the device. Although the electrostatic force actuation is used throughout the disclosure as an example approach for actuating of the disclosed MEMS structures, the technology described herein is not limited to electrostatic actuation, and the MEMS structures can therefore be used with any form of actuation in accordance with the technology disclosed herein.

In various embodiments, the PCB 207 includes a plurality of electrical inputs and outputs wherein each of the plurality of electrical inputs and outputs provides an electrical connection between an external source and to the one or more electrodes of the integrated device 200. The physical architecture of the assembly requires electrical connections permitting discrete signal transmission to different components in the integrated device 200 from an external source. This electrical signal I/O can be used for the capture of discrete biosystems within the fluidic cavity 162 as well as the control of a component, e.g., the actuator stage 130, within the MEMS cavity 122, for example. Additional electrical signal I/O can be used for various sensing functions, such as the detection of changes in actuator impedance for position measurement, for example, serving a variety of functions depending on the application. These electrical connections may comprise a variety of configurations, such as through-chip vias (e.g., through-silicon-via), in-plane routing within the 2D area of a functional layer (“interconnects”), shallow vias within the membrane portion 110, an added interposer portion, (e.g., interposer 208), such as an electrical redistribution portion and/or an application-specific integrated circuit (ASIC) used to physically map the electrical signal I/O between different electrical contact layouts, or a combination or two or more of these configurations. In various embodiments, a combination of all of these methods whereby the electrical signal I/O is all routed to a common surface for “mounting” to a ball grid array (BGA) or other surface mount for PCB bonding or socketing during packaging.

This electrical conduction architecture can have particular characteristics that permit the functional operation of the integrated device 200 and can be specially insulated or electrically isolated in some cases depending on the application. Material and geometrical characteristics of the conductive and insulating elements are carefully controlled. Additionally, material selection that permits adequate bonding of each portion or layer permitting successful electrical connectivity between the chip, interposer portion, any other bonded component such as an integrated control ASIC, and surface mount or socket can be implemented, in accordance with various embodiments.

In various embodiments, the integrated device 200 further includes one or more through-silicon-vias (TSV) for electrical interconnection between a MEMS portion, such as MEMS portion 120, of the integrated device, such as the integrated device 100 or 200, and the PCB 207.

In various embodiments, the chip 220 includes electrical signal interfacing with control elements (electrical systems that are used to close the control loop between device sensing, device actuation, and external input) that can either be directly integrated (a bonded ASIC) or off-chip. In various embodiments, the chip 220 can be mounted via an interposer 208, as described above, that is then mounted to the PCB 207 either through a reversible connection such as a socket or an irreversible connection such as a BGA, illustrated in FIG. 2B. The BGA or socket may have many 2D configurations depending on the electrical signal input/output (I/O) density and layout of the chip 220. In various embodiments, the electrical signal I/O is routed off of the chip 220 through the PCB 207 to electrical components and logic elements referred to as “off-chip logic”. In some embodiments, an operation of the integrated device 200 may include open- and closed-loop control by a system capable of executing predetermined programs. This control system can either be an integrated CMOS ASIC directly bonded onto the chip, or it may be an off-shelf component that resides on a PCB to which the chip has its electrical signal 110 routed, for example.

In various embodiments, the integrated device 200 further includes one or more electrodes, such as the capture-site electrodes 180, for trapping one or more particles (such as, the nanoscale or microscale materials 165) in the fluidic cavity 260, as illustrated in a zoomed-in portion 200a of FIG. 2A. In various embodiments, the one or more capture-site electrodes, such as the capture-site electrodes 180, can be operated via electrical interconnections provided via a surface routing between the integrated device 200 (i.e., the LOC) and the PCB 207.

As illustrated in FIG. 2A, the integrated package further includes a gasket 205 disposed on edges of the integrated device 200. In various embodiments, the gasket 205 hermetically seals the fluidic cavity 260. In various embodiments, the gasket 205 fluidically seals the fluidic cavity 260. In various embodiments, the gasket 205 is disposed in-plane with the integrated device 200 (i.e., the LOC) and conforms to edges of the LOC.

In various embodiments, the LOC system (i.e., the integrated package) includes a plurality of integrated devices, such as the integrated device 100 or the integrated device 200). In such embodiments, the fluidic cap 270 can include a fluidic inlet and a fluidic outlet for each of the plurality of integrated devices 100 or 200.

FIG. 2B shows a schematic illustration of a process flow 30 for packaging the integrated device 200, for example, to obtain an integrated package, in accordance with various embodiments. As illustrated in FIG. 2B, the integrated device 200 is bonded to a substrate, for example, such as interposer 201. In various embodiments, the integrated device 200 can have a thickness between about 0.01 mm and about 10.0 mm. In various embodiments, the integrated device 200 can have a thickness between about 0.05 mm and about 8 mm, between about 0.1 mm and about 6 mm, between about 0.2 mm and about 5 mm, between about 0.5 mm and about 4 mm, between about 1 mm and about 3 mm, between about 0.01 mm and about 2 mm, between about 0.05 mm and about 5 mm, between about 0.1 mm and about 2 mm, or between about 0.1 mm and about 3 mm, inclusive of any thicknesses or ranges of thickness therebetween.

In various embodiments, the interposer 201 can have a thickness between about 0.1 μm and about 5.0 mm. In various embodiments, the interposer 201 can have a thickness between about 0.2 μm and about 4.5 mm, between about 0.3 μm and about 4.0 mm, between about 0.5 μm and about 3.5 mm, between about 1 μm and about 3.0 mm, between about 10 μm and about 3.5 mm, between about 50 μm and about 3.0 mm, between about 100 μm and about 2.5 mm, between about 250 μm and about 2.0 mm, between about 0.5 mm and about 1.5 mm, between about 0.8 mm and about 1.5 mm, or between about 1.0 mm and about 5.0 mm, inclusive of any thicknesses or ranges of thickness therebetween.

As illustrated in FIG. 2B, the PCB 207 is bonded to the interposer 201 via one or more solder balls 206. In order to achieve uniformity in bonding, a ball grid array of the solder balls 206 can be used to bond the PCB 207 to the interposer 201, which is illustrated as already attached to the integrated device 200. In other words, the integrated package further includes a ball grid array for bonding of the integrated device 200 to the PCB 207. In various embodiments, the ball grid array provides a surface mount for bonding an array of contact pads between the integrated device 200 and the PCB 207 with beads of solder and flux. In various embodiments, the ball grid array can include any reasonable array ranges in any pattern or format, including for example, in a rectangular, square, any grid or circular arrangement as suitable.

As further illustrated in FIG. 2B, the process flow 30 includes assembling a driver IC and a surface mounted device (SMD) 40 on the PCB 207.

Furthermore, FIG. 2B illustrates assembling the fluidic cap 270 having two fluidic vias that are fluidically coupled to outer microchannels 50. In accordance with various embodiments, the microchannels 50 are designed to input a fluidic medium containing nanoscale or microscale materials, such as nanoscale or microscale materials 165 of FIGS. 1A and 1B into the integrated device 200.

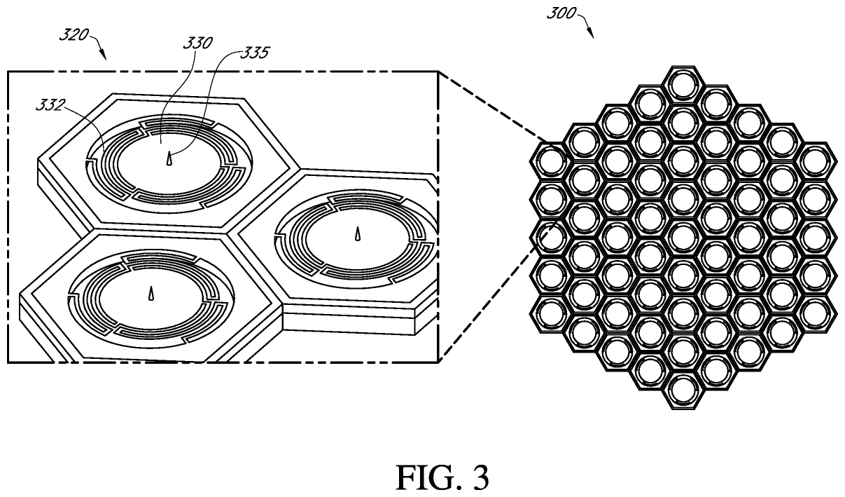

FIG. 3 shows a schematic view of an array of the example MEMS portion 320 of an integrated device 300, according to various implementations. As shown in FIG. 3, the array the example MEMS portion 320 includes a plurality of actuator stages 330 that are arranged in a hexagonal tile. In various embodiments, the plurality of actuator stages 330 may be arranged in a rectangular grid array, a square grid array, or any suitable grid array.

In various implementations of the integrated device 300, the plurality of actuator stages 330 can be in fluidic communication. For example, each of the plurality of actuator stages 330 can be configured as a unit cell (e.g., the unit cell 120a described with respect to FIG. 1A) and each of the unit cells 120a are fluidically interconnected, for example, via the MEMS fluidic access via 126 described with respect to FIG. 1A. In accordance with various embodiments, the fluid contained within a MEMS cavity of the unit cell 120a (e.g., the MEMS cavity 122 used to provide fluidic interconnection) includes for example, but limited to, an aqueous fluid, an aqueous buffer, an organic solvent, a hydrophobic fluid, a gas, aqueous solution containing biological or chemical reagents, organic solvents, mineral oil, fluorinated oil, air, mixed gases for cell culture (e.g., 5% CO₂), inert gas, and the like.

In various implementations of the integrated device 300, the plurality of actuator stages 330 can range from about 1 to about 10⁸ actuator stages. According to various implementations, each of the plurality of actuator stages 330 in the integrated device 300 is supported by two or more actuator arms 332. According to various implementations, each of the plurality of actuator stages 330 in the integrated device 300 includes a respective sharp member 335. In various implementations, the plurality of actuator stages 330 are separated from each other, e.g., center to center distance between two adjacent actuator stages 330, by about 0.1 μm and 10 cm, about 0.1 μm and 1 cm, about 0.1 μm and 1 mm, about 0.1 μm and 500 μm, about 0.1 μm and 100 μm, about 0.1 μm and 75 μm, about 0.1 μm and 50 μm, about 0.1 μm and 25 μm, about 0.1 μm and 10 μm, about 1 μm and 10 cm, about 1 μm and 1 cm, about 1 μm and 1 mm, about 1 μm and 500 μm, about 1 μm and 100 μm, about 1 μm and 75 μm, about 1 μm and 50 μm, about 1 μm and 25 μm, about 1 μm and 10 μm, about 10 μm and 10 cm, about 10 μm and 1 cm, about 10 μm and 1 mm, about 10 μm and 500 μm, about 10 μm and 200 μm, about 10 μm and 100 μm, about 10 μm and 75 μm, about 10 μm and 50 μm, about 10 μm and 25 μm, about 10 μm and 1 mm, or about 20 μm and 1 mm, inclusive of any separation distance ranges therebetween.

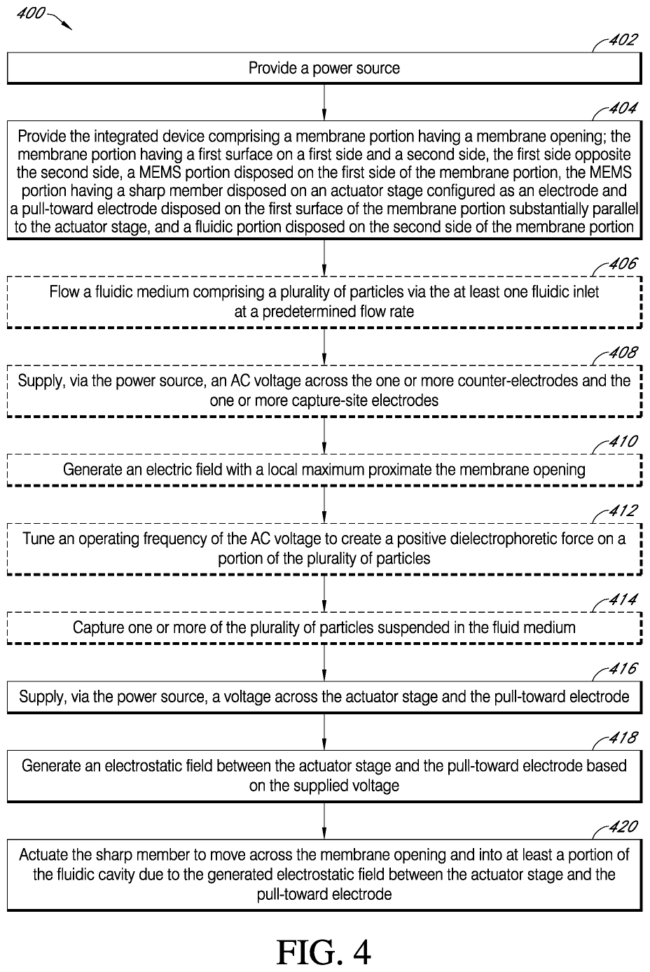

FIG. 4 is a flow chart for a method 400 of operating an integrated device, according to an illustrative implementation. The method 400 includes providing a power source, at step 402 and providing the integrated device, at step 404. In various embodiments, the integrated device is substantially similar or identical to the integrated device 100 or the integrated device 200. The integrated device includes a membrane portion having a membrane opening; the membrane portion having a first surface on a first side and a second side, the first side opposite the second side, a MEMS portion disposed on the first side of the membrane portion, the MEMS portion having a sharp member disposed on an actuator stage configured as an electrode and a pull-toward electrode disposed on the first surface of the membrane portion substantially parallel to the actuator stage, and a fluidic portion disposed on the second side of the membrane portion.

In accordance with various implementations, the method 400 optionally includes, at step 406, flowing a fluidic medium comprising a plurality of particles via the at least one fluidic inlet at a predetermined flow rate. In accordance with various implementations, the method 400 optionally includes, at step 408, supplying, via the power source, an AC voltage across the one or more counter-electrodes and the one or more capture-site electrodes. In accordance with various implementations, the method 400 optionally includes, at step 410, generating an electric field with a local maximum proximate the membrane opening; at step 412, tuning an operating frequency of the AC voltage to create a positive dielectrophoretic force on a portion of the plurality of particles; and at step 414, capturing one or more of the plurality of particles in the fluid medium.

As illustrated in FIG. 4, the method 400 includes, at step 416, supplying, via the power source, a voltage across the actuator stage and the pull-toward electrode. The method 400 includes generating an electrostatic field between the actuator stage and the pull-toward electrode based on the supplied voltage, at step 418, and actuating the sharp member to move across the membrane opening and into at least a portion of the fluidic cavity due to the generated electrostatic field between the actuator stage and the pull-toward electrode, at step 420. In various embodiments, the fluidic portion comprises a fluidic cap forming a fluidic cavity in the fluidic portion, the fluidic cap having a surface thereon, at least one fluidic inlet, and at least one fluidic outlet.

In various embodiments, the second side of the membrane portion comprises one or more capture-site electrodes disposed on a second surface of the membrane portion and adjacent to the membrane opening. In various embodiments, the one or more capture-site electrodes are electrically connected to form a circular capture-site electrode. In various embodiments, two or more capture-site electrodes of the one or more capture-site electrodes are used partially as a phased sensor array. In various embodiments, the one or more capture-site electrodes are a pair of bi-polar electrodes that are disposed across the membrane opening.

In various embodiments, the fluidic cap comprises one or more counter-electrodes disposed on the surface of the fluidic cap across from the membrane opening.

In various embodiments, the tuning of the operating frequency of the AC voltage comprises determining a competing effect of dielectrophoresis (DEP) force induced by the applied AC voltage with respect to a hydrodynamic force exerted upon one or more particles of the flowing fluidic medium. In various embodiments, the capturing one or more of the plurality of particles via the DEP force comprises supplying sufficient DEP force to capture the one or more particles proximate to the membrane opening by overcoming the hydrodynamic force on the one or more particles flowing in the fluid medium.

In various embodiments, the method 400 optionally includes adjusting the AC voltage such that the DEP force is tuned to capture a single particle. In various embodiments, the method 400 optionally includes interrogating the single particle by the sharp member. In various embodiments, a tip of the sharp member is configured to deliver a payload to the single particle.

In various embodiments, the one or more pull-toward electrodes are disposed adjacent to the membrane opening and/or at least partially surrounding the membrane opening. In various embodiments, the one or more pull-toward electrodes are disposed partially bracketing the membrane opening.

In various embodiments, at least partially surrounding the membrane opening refers to surrounding the membrane opening in at least one dimension. In various embodiments, at least partially surrounding the membrane opening refers to substantially enclosing the membrane opening in at least one dimension. In various embodiments, the one or more pull-toward electrodes are electrically connected to form a circular pull-toward electrode.

In various embodiments, the second side of the membrane portion comprises at least a portion of one or more capture-site electrodes partially or fully embedded in the second side of the membrane portion and adjacent to the membrane opening. In various embodiments, the one or more capture-site electrodes comprises a circular capture-site electrode geometry or an annular capture-site electrode geometry. In various embodiments, the one or more capture-site electrodes are a pair of bi-polar electrodes that are disposed across the membrane opening. In various embodiments, the one or more counter-electrodes and the one or more capture-site electrodes are configured as one or more electrode pairs. In various embodiments, the one or more electrode pairs are configured to capture one or more particles in the fluid medium. In various embodiments, the one or more particles are captured using dielectrophoretic force. In various embodiments, the dielectrophoretic force is applied perpendicular to the membrane opening.

In various embodiments, the actuator stage is suspended within a MEMS cavity of the MEMS portion and supported by two or more actuator arms attached to walls of the MEMS cavity. In various embodiments, the two or more actuator arms comprises a serpentine pattern. In various embodiments, the two or more actuator arms comprises a conductive or sufficiently conductive material. In various embodiments, the actuator stage is suspended a predetermined distance away from the first surface of the membrane portion, and in operation, the actuator stage moves relative to the first surface of the membrane portion.

In various embodiments, a fluid medium flows from the at least one fluidic inlet to the at least one fluidic outlet at a predetermined flow rate. In various embodiments, the sharp member comprises silicon. In various embodiments, wherein the sharp member is coated with one or more materials configured for conjugation of a polynucleotide to the sharp member. In various embodiments, at least a portion of the sharp member is coated with a plurality of gold atoms. In various embodiments, the sharp member coated with gold atoms is capable of being attached to by one or more biological molecules.

In various embodiments, the sharp member comprises at least one of a Langmuir-Bodgett film, a functionalized glass, germanium, PTFE, polystyrene, gallium arsenide, silver, nylon, PVP, silicon oxide, metal oxide, or ceramics. In various embodiments, the sharp member is conjugated to a polynucleotide via a covalent bond, a thiol (—SH) modifier, avidin/biotin coupling chemistry, or a mediating linker molecule from one of polyethylene glycol (PEG) or polyethyleneimine (PEI). In various embodiments, the sharp member is capable of carrying a payload from a list of nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs, exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs. In various embodiments, the sharp member is capable of carrying a genetic material capable of stably integrating into a cell's genome from a list of gRNA comprising crRNA or a tracrRNA capable of complexing with a Cas protein or plasmid expressing Cas protein, TALENs or zinc finger nucleases.

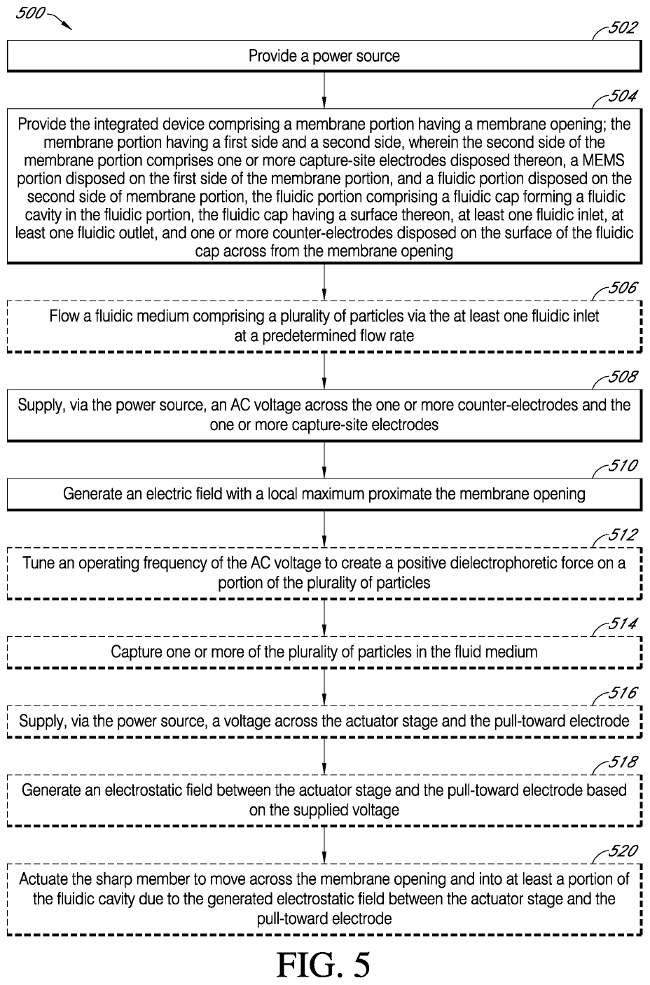

FIG. 5 is a flow chart for a method 500 of operating an integrated device, according to an illustrative implementation. The method 500 includes providing a power source, at step 502 and providing the integrated device, at step 504. In various embodiments, the integrated device is substantially similar or identical to the integrated device 100 or the integrated device 200. The integrated device includes a membrane portion having a membrane opening; the membrane portion having a first side and a second side, wherein the second side of the membrane portion comprises one or more capture-site electrodes disposed thereon, a MEMS portion disposed on the first side of the membrane portion, and a fluidic portion disposed on the second side of the membrane portion, the fluidic portion comprising a fluidic cap forming a fluidic cavity in the fluidic portion, the fluidic cap having a surface thereon, at least one fluidic inlet, at least one fluidic outlet, and one or more counter-electrodes disposed on the surface of the fluidic cap across from the membrane opening.

In accordance with various implementations, the method 500 optionally includes, at step 506, flowing a fluidic medium comprising a plurality of particles via the at least one fluidic inlet at a predetermined flow rate.

In accordance with various implementations, the method 500 further includes at step 508, supplying, via the power source, an AC voltage across the one or more counter-electrodes and the one or more capture-site electrodes. In accordance with various implementations, the method 500 further includes at step 510, generating an electric field with a local maximum proximate the membrane opening.

In accordance with various implementations, the method 500 optionally includes, at step 512, tuning an operating frequency of the AC voltage to create a positive dielectrophoretic force on a portion of the plurality of particles; and at step 514, capturing one or more of the plurality of particles in the fluid medium.

In various embodiments, the second side of the membrane portion comprises at least a portion of the one or more capture-site electrodes partially or fully embedded in the second side of the membrane portion and adjacent to the membrane opening. In various embodiments, the one or more capture-site electrodes are disposed adjacent to the membrane opening and comprises a circular capture-site electrode geometry or an annular capture-site electrode geometry. In various embodiments, the one or more capture-site electrodes are a pair of bi-polar electrodes that are disposed across the membrane opening. In various embodiments, the one or more counter-electrodes and the one or more capture-site electrodes are configured as one or more electrode pairs.

In various embodiments, the MEMS portion comprises a sharp member disposed on an actuator stage within a MEMS cavity. In various embodiments, the first side of the membrane portion comprises one or more pull-toward electrodes disposed on the first surface of the membrane portion and adjacent to the membrane opening and/or at least partially surrounding the membrane opening. In various embodiments, the one or more pull-toward electrodes are disposed partially bracketing the membrane opening. In various embodiments, at least partially surrounding the membrane opening refers to surrounding the membrane opening in at least one dimension. In various embodiments, at least partially surrounding the membrane opening refers to substantially enclosing the membrane opening in at least one dimension. In various embodiments, the one or more pull-toward electrodes are electrically connected to form a circular pull-toward electrode. In various embodiments, the actuator stage is configured as an electrode and is substantially parallel to the one or more pull-toward electrodes.

In various embodiments, the tuning of the operating frequency of the AC voltage comprises determining a competing effect of dielectrophoresis (DEP) force induced by the applied AC voltage with respect to a hydrodynamic force exerted upon one or more particles of the flowing fluidic medium. In various embodiments, the capturing one or more of the plurality of particles via the DEP force comprises supplying sufficient DEP force to capture the one or more particles proximate to the membrane opening by overcoming the hydrodynamic force on the one or more particles flowing in the fluid medium.

In accordance with various implementations, the method 500 optionally includes at step 516, supplying, via the power source, a voltage across the actuator stage and the one or more pull-toward electrodes; at step 518, generating an electrostatic field between the actuator stage and the one or more pull-toward electrodes based on the supplied voltage; and at step 520, actuating the sharp member to move across the membrane opening and into at least a portion of the fluidic cavity based on the generated electrostatic field between the actuator stage and the one or more pull-toward electrodes.

In accordance with various implementations, the method 500 optionally includes adjusting the AC voltage such that the DEP force is tuned to capture a single particle. In accordance with various implementations, the method 500 optionally includes interrogating the single particle by the sharp member.

In various embodiments, a tip of the sharp member is configured to deliver a payload to the single particle. In various embodiments, the one or more captured particles are interrogated by the sharp member when actuated. In various embodiments, a tip of the sharp member is configured to deliver a payload to the one or more captured particles.

In various embodiments, the second side of the membrane portion comprises at least a portion of one or more capture-site electrodes partially or fully embedded in the second side of the membrane portion and adjacent to the membrane opening. In various embodiments, the one or more capture-site electrodes comprises a circular capture-site electrode geometry or an annular capture-site electrode geometry. In various embodiments, the one or more capture-site electrodes are a pair of bi-polar electrodes that are disposed across the membrane opening.

In various embodiments, the actuator stage is suspended within the MEMS cavity of the MEMS portion and supported by two or more actuator arms attached to walls of the MEMS cavity. In various embodiments, the two or more actuator arms comprises a serpentine pattern. In various embodiments, the two or more actuator arms comprises a conductive material. In various embodiments, the actuator stage is suspended a predetermined distance away from the first surface of the membrane portion, and in operation, the actuator stage moves relative to the first surface of the membrane portion.

In various embodiments, the sharp member comprises silicon. In various embodiments, the sharp member is coated with one or more materials configured for conjugation of a polynucleotide to the sharp member. In various embodiments, at least a portion of the sharp member is coated with a plurality of gold atoms. In various embodiments, the sharp member coated with gold atoms is capable of being attached to one or more biological molecules. In various embodiments, the sharp member comprises at least one of a Langmuir-Bodgett film, a functionalized glass, germanium, PTFE, polystyrene, gallium arsenide, silver, membrane, nylon, PVP, silicon oxide, metal oxide, or ceramics. In various embodiments, the sharp member is conjugated to a polynucleotide via a covalent bond, a thiol (—SH) modifier, avidin/biotin coupling chemistry, or a mediating linker molecule from one of polyethylene glycol (PEG) or polyethyleneimine (PEI). In various embodiments, the sharp member is capable of carrying a payload from a list of nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs (e.g., cell membrane), exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs. In various embodiments, the sharp member is capable of carrying a genetic material capable of stably integrating into a cell's genome from a list of gRNA comprising crRNA or a tracrRNA capable of complexing with a Cas protein or plasmid expressing Cas protein, TALENs or zinc finger nucleases.

### Recitation of Embodiments

Embodiment 1. An integrated device comprising: a membrane portion having a membrane opening; the membrane portion having a first side and a second side, the first side opposite the second side; a MEMS portion disposed on the first side of the membrane portion, the MEMS portion having a sharp member disposed on an actuator stage within a MEMS cavity, the sharp member having a distal (or base) portion attached substantially perpendicular to the actuator stage; and a fluidic portion disposed on the second side of the membrane portion, the fluidic portion having a fluidic cavity for flowing a fluid medium within the fluidic portion, wherein the membrane opening provides access between the MEMS portion and the fluidic portion and is substantially aligned with a proximal portion of the sharp member, and in operation, the proximal portion of the sharp member moves across the membrane opening and into at least a portion of the fluidic cavity.

Embodiment 2. The integrated device of Embodiment 1, wherein the first side of the membrane portion comprises one or more pull-toward electrodes disposed on a first surface of the membrane portion and adjacent to the membrane opening and/or at least partially surrounding the membrane opening.

Embodiment 3. The integrated device of any one of Embodiments 1 or 2, wherein the one or more pull-toward electrodes comprises a sheet of electrode material or the adjacent to the membrane opening comprises the one or more pull-toward electrodes that are disposed partially bracketing the membrane opening.

Embodiment 4. The integrated device of any one of Embodiments 1-3, wherein at least partially surrounding the membrane opening comprises surrounding the membrane opening in at least one dimension.

Embodiment 5. The integrated device of any one of Embodiments 1-4, wherein at least partially surrounding the membrane opening comprises substantially enclosing the membrane opening in at least one dimension.

Embodiment 6. The integrated device of any one of Embodiments 1-5, wherein the one or more pull-toward electrodes are electrically connected to form a circular pull-toward electrode.

Embodiment 7. The integrated device of any one of Embodiments 1-6, wherein the second side of the membrane portion comprises at least a portion of one or more capture-site electrodes partially or fully embedded in the second side of the membrane portion and adjacent to the membrane opening.

Embodiment 8. The integrated device of any one of Embodiments 1-7, wherein the second side of the membrane portion comprises one or more capture-site electrodes disposed on a second surface of the membrane portion and adjacent to the membrane opening.

Embodiment 9. The integrated device of any one of Embodiments 1-8, wherein the one or more capture-site electrodes comprises a circular capture-site electrode geometry or an annular capture-site electrode geometry.

Embodiment 10. The integrated device of any one of Embodiments 1-9, wherein the one or more capture-site electrodes are a pair of bi-polar electrodes that are disposed across the membrane opening.

Embodiment 11. The integrated device of any one of Embodiments 1-10, wherein the fluidic portion comprises one or more counter-electrodes disposed on a fluidic cavity surface across from the membrane opening, and in operation, the one or more counter-electrodes and the one or more capture-site electrodes are configured as one or more electrode pairs.

Embodiment 12. The integrated device of any one of Embodiments 1-11, wherein the one or more electrode pairs are configured to capture one or more particles in the fluid medium.

Embodiment 13. The integrated device of any one of Embodiments 1-12, wherein the one or more particles are captured using dielectrophoretic force.

Embodiment 14. The integrated device of any one of Embodiments 1-13, wherein the dielectrophoretic force is applied perpendicular to the membrane opening.

Embodiment 15. The integrated device of any one of Embodiments 1-14, wherein the actuator stage is suspended within the MEMS cavity and supported by two or more actuator arms attached to walls of the MEMS cavity, wherein the two or more actuator arms comprises at least a serpentine pattern or a conductive material comprising at least one of single crystal silicon, polycrystalline silicon, nanocrystalline silicon, amorphous silicon, hydrogenated amorphous silicon, a metal, a metallic alloy, a eutectic, a ceramic, a composite, a polymer, a doped silicon, an allotrope of silicon, an inorganic glassy material or mixture, an inorganic polycrystalline material or mixture, an inorganic single crystalline material or mixture, a ceramic material comprising metal oxides, metalloid oxides, metal or metalloid nitrides, metal or metalloid oxides with nitrogen or other non-metalloid or metal elements, a doped combination of the above materials, any layered stack or structural combination of the above materials.

Embodiment 16. The integrated device of any one of Embodiments 1-15, wherein the actuator stage is suspended a predetermined distance away from the first surface of the membrane portion, and in operation, the actuator stage moves relative to the first surface of the membrane portion.

Embodiment 17. The integrated device of any one of Embodiments 1-16, wherein the MEMS cavity is configured to contain a fluid within the MEMS cavity.

Embodiment 18. The integrated device of any one of Embodiments 1-17, wherein the fluid disposed within the MEMS cavity is miscible with the fluid medium.

Embodiment 19. The integrated device of any one of Embodiments 1-18, wherein the fluid disposed within the MEMS cavity is immiscible with the fluid medium.

Embodiment 20. The integrated device of any one of Embodiments 1-19, wherein the sharp member is disposed on a first surface of the actuator stage and the MEMS cavity comprises one or more pull-away electrodes disposed on a MEMS cavity surface facing a second surface of the actuator stage.

Embodiment 21. The integrated device of any one of Embodiments 1-20, wherein the fluidic portion comprises a fluidic inlet and a fluidic outlet and the fluid medium flows from the fluidic inlet to the fluidic outlet at a predetermined flow rate.

Embodiment 22. The integrated device of any one of Embodiments 1-21, wherein the fluidic portion comprises a plurality of fluidic inlets and a plurality of fluidic outlets.

Embodiment 23. The integrated device of any one of Embodiments 1-22, wherein the integrated device is electrically coupled to a power source.

Embodiment 24. The integrated device of any one of Embodiments 1-23, wherein the sharp member comprises silicon.

Embodiment 25. The integrated device of any one of Embodiments 1-24, wherein the sharp member is coated with one or more materials configured for conjugation of a polynucleotide to the sharp member.

Embodiment 26. The integrated device of any one of Embodiments 1-25, wherein at least a portion of the sharp member is coated with a plurality of gold atoms, and wherein the sharp member coated with gold atoms is capable of being attached to one or more biological molecules.

Embodiment 27. The integrated device of any one of Embodiments 1-26, wherein the sharp member comprises at least one of a Langmuir-Bodgett film, a functionalized glass, germanium, PTFE, polystyrene, gallium arsenide, silver, membrane, nylon, PVP, silicon oxide, metal oxide, or ceramics.

Embodiment 28. The integrated device of any one of Embodiments 1-27, wherein the sharp member comprises one or more bond types including at least one of a covalent bond, an ionic bond, an electrostatic bond, a hydrogen bond, or a van der waals bond or is conjugated to a polynucleotide via a covalent bond, a thiol (—SH) modifier, avidin/biotin coupling chemistry, or a mediating linker molecule from one of polyethylene glycol (PEG) or polyethyleneimine (PEI).

Embodiment 29. The integrated device of any one of Embodiments 1-28, wherein the sharp member is capable of carrying one or more payloads from a list of nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs, exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs.

Embodiment 30. The integrated device of any one of Embodiments 1-29, wherein the sharp member is capable of carrying a genetic material capable of stably integrating into a cell's genome from a list of gRNA comprising crRNA or a tracrRNA capable of complexing with a Cas protein or plasmid expressing Cas protein, TALENs or zinc finger nucleases.

Embodiment 31. An integrated package comprising: a substrate; and a lab-on-chip (LOC) disposed on the substrate, the LOC comprising at least one integrated device comprising: a membrane portion having a membrane opening; the membrane portion having a first side and a second side, the first side opposite the second side, a MEMS portion disposed on the first side of the membrane portion, the MEMS portion having a sharp member disposed on an actuator stage within a MEMS cavity, and a fluidic portion disposed on the second side of the membrane portion, the fluidic portion having a fluidic cavity for flowing a fluid medium within the fluidic portion; and a fluidic cap forming a surface of the fluidic portion of the LOC, the fluidic cap having a fluidic inlet and a fluidic outlet.

Embodiment 32. The integrated package of Embodiment 31, wherein the substrate comprises at least one of a printed circuit board (PCB), a composite including at least a glass epoxy composite, or a ceramic including at least one of a ceramic composite, the at least one integrated device further comprising: an interposer disposed between the LOC and the substrate.

Embodiment 33. The integrated package of any one of Embodiments 31 or 32, wherein the interposer comprises an electrical redistribution layer, an application-specific integrated circuit (ASIC) configured to physically map electrical signal inputs and outputs between different electrical contact layouts, or a combination thereof.

Embodiment 34. The integrated package of any one of Embodiments 31-33, wherein the LOC comprises one or more electrodes for providing an electrostatic force used for actuating the actuator stage.

Embodiment 35. The integrated package of any one of Embodiments 31-34, wherein the actuator stage is actuated via an applied electrostatic force.

Embodiment 36. The integrated package of any one of Embodiments 31-35, wherein the substrate comprises a plurality of electrical inputs and outputs wherein each of the plurality of electrical inputs and outputs provides an electrical connection between an external source and to the one or more electrodes of the LOC.

Embodiment 37. The integrated package of any one of Embodiments 31-36, wherein the LOC further comprises a through-silicon-via (TSV) for electrical interconnection between the MEMS portion and the substrate.

Embodiment 38. The integrated package of any one of Embodiments 31-37, wherein the LOC comprises one or more electrodes for trapping one or more particles in the fluidic cavity via one or more capture-site electrodes.

Embodiment 39. The integrated package of any one of Embodiments 31-38, wherein the one or more capture-site electrodes is operated via electrical interconnections provided via a surface routing between the LOC and the substrate.

Embodiment 40. The integrated package of any one of Embodiments 31-39, further comprising: a gasket disposed on edges of the LOC.

Embodiment 41. The integrated package of any one of Embodiments 31-40, wherein the gasket hermetically seals the fluidic cavity.

Embodiment 42. The integrated package of any one of Embodiments 31-41, wherein the gasket fluidically seals the fluidic cavity.

Embodiment 43. The integrated package of any one of Embodiments 31-42, wherein the gasket is disposed in-plane with the LOC and conforms to edges of the LOC.

Embodiment 44. The integrated package of any one of Embodiments 31-43, further comprising: a ball grid array for bonding of the LOC to the substrate, wherein the ball grid array provides a surface mount for bonding an array of contact pads between the LOC and the substrate with beads of solder and flux.

Embodiment 45. The integrated package of any one of Embodiments 31-44, wherein the LOC comprises a plurality of integrated devices, wherein the fluidic cap comprises a fluidic inlet and a fluidic outlet for each of the plurality of integrated devices.

Embodiment 46. The integrated package of any one of Embodiments 31-45, wherein the plurality of integrated devices comprises different capture-side electrode geometries within the same LOC, or wherein the plurality of integrated devices comprises at least one of annular capture-side electrode and at least a bipolar capture side electrode geometry.

Embodiment 47. The integrated package of any one of Embodiments 31-46, wherein the first side of the membrane portion comprises one or more pull-toward electrodes disposed on a first surface of the membrane portion and adjacent to the membrane opening and/or at least partially surrounding the membrane opening.

Embodiment 48. The integrated package of any one of Embodiments 31-47, wherein the one or more pull-toward electrodes comprises a sheet of electrode material or the adjacent to the membrane opening comprises the one or more pull-toward electrodes are disposed partially bracketing the membrane opening.

Embodiment 49. The integrated package of any one of Embodiments 31-48, wherein at least partially surrounding the membrane opening comprises surrounding the membrane opening in at least one dimension.

Embodiment 50. The integrated package of any one of Embodiments 31-49, wherein at least partially surrounding the membrane opening comprises substantially enclosing the membrane opening in at least one dimension.

Embodiment 51. The integrated package of any one of Embodiments 31-50, wherein the one or more pull-toward electrodes are electrically connected to form a circular pull-toward electrode.

Embodiment 52. The integrated package of any one of Embodiments 31-51, wherein the second side of the membrane portion comprises at least a portion of one or more capture-site electrodes partially or fully embedded in the second side of the membrane portion and adjacent to the membrane opening.

Embodiment 53. The integrated package of any one of Embodiments 31-52, wherein the second side of the membrane portion comprises one or more capture-site electrodes disposed on a second surface of the membrane portion and adjacent to the membrane opening.

Embodiment 54. The integrated package of any one of Embodiments 31-53, wherein the one or more capture-site electrodes comprises a circular capture-site electrode geometry or an annular capture-site electrode geometry.

Embodiment 55. The integrated package of any one of Embodiments 31-54, wherein the one or more capture-site electrodes are a pair of bi-polar electrodes that are disposed across the membrane opening.

Embodiment 56. The integrated package of any one of Embodiments 31-55, wherein the fluidic portion comprises one or more counter-electrodes disposed on a fluidic cavity surface across from the membrane opening, and in operation, the one or more counter-electrodes and the one or more capture-site electrodes are configured as one or more electrode pairs.

Embodiment 57. The integrated package of any one of Embodiments 31-56, wherein the one or more electrode pairs are configured to capture one or more particles in the fluid medium.

Embodiment 58. The integrated package of any one of Embodiments 31-57, wherein the one or more particles are captured using dielectrophoretic force.

Embodiment 59. The integrated package of any one of Embodiments 31-58, wherein the dielectrophoretic force is applied perpendicular to the membrane opening.

Embodiment 60. The integrated package of any one of Embodiments 31-59, wherein the actuator stage is suspended within the MEMS cavity and supported by two or more actuator arms attached to walls of the MEMS cavity, wherein the two or more actuator arms comprises at least a serpentine pattern or a conductive material comprising at least one of single crystal silicon, polycrystalline silicon, nanocrystalline silicon, amorphous silicon, hydrogenated amorphous silicon, a metal, a metallic alloy, a eutectic, a ceramic, a composite, a polymer, a doped silicon, an allotrope of silicon, an inorganic glassy material or mixture, an inorganic polycrystalline material or mixture, an inorganic single crystalline material or mixture, a ceramic material comprising metal oxides, metalloid oxides, metal or metalloid nitrides, metal or metalloid oxides with nitrogen or other non-metalloid or metal elements, a doped combination of the above materials, any layered stack or structural combination of the above materials.

Embodiment 61. The integrated package of any one of Embodiments 31-60, wherein the actuator stage is suspended a predetermined distance away from the first surface of the membrane portion, and in operation, the actuator stage moves relative to the first surface of the membrane portion.

Embodiment 62. The integrated package of any one of Embodiments 31-61, wherein the MEMS cavity is configured to contain a fluid within the MEMS cavity.

Embodiment 63. The integrated package of any one of Embodiments 31-62, wherein the fluid disposed within the MEMS cavity is miscible with the fluid medium.

Embodiment 64. The integrated package of any one of Embodiments 31-63, wherein the fluid disposed within the MEMS cavity is immiscible with the fluid medium.

Embodiment 65. The integrated package of any one of Embodiments 31-64, wherein the sharp member is disposed on a first surface of the actuator stage and the MEMS cavity comprises one or more pull-away electrodes disposed on a MEMS cavity surface facing a second surface of the actuator stage.

Embodiment 66. The integrated package of any one of Embodiments 31-65, wherein the fluidic portion comprises a fluidic inlet and a fluidic outlet and the fluid medium flows from the fluidic inlet to the fluidic outlet at a predetermined flow rate.

Embodiment 67. The integrated package of any one of Embodiments 31-66, wherein the fluidic portion comprises a plurality of fluidic inlets and a plurality of fluidic outlets.

Embodiment 68. The integrated package of any one of Embodiments 31-67, wherein the integrated device is electrically coupled to a power source.

Embodiment 69. The integrated package of any one of Embodiments 31-68, wherein the sharp member comprises silicon or is coated with one or more materials configured for conjugation of a polynucleotide to the sharp member.

Embodiment 70. The integrated package of any one of Embodiments 31-69, wherein at least a portion of the sharp member is coated with a plurality of gold atoms, and wherein the sharp member coated with gold atoms is capable of being attached to one or more biological molecules.

Embodiment 71. The integrated package of any one of Embodiments 31-70, wherein the sharp member comprises at least one of a Langmuir-Bodgett film, a functionalized glass, germanium, PTFE, polystyrene, gallium arsenide, silver, membrane, nylon, PVP, silicon oxide, metal oxide, or ceramics.

Embodiment 72. The integrated package of any one of Embodiments 31-71, wherein the sharp member comprises one or more bond types including at least one of a covalent bond, an ionic bond, an electrostatic bond, a hydrogen bond, or a van der waals bond or is conjugated to a polynucleotide via a covalent bond, a thiol (—SH) modifier, avidin/biotin coupling chemistry, or a mediating linker molecule from one of polyethylene glycol (PEG) or polyethyleneimine (PEI).

Embodiment 73. The integrated package of any one of Embodiments 31-72, wherein the sharp member is capable of carrying one or more payloads from a list of nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs, exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs.

Embodiment 74. The integrated package of any one of Embodiments 31-73, wherein the LOC comprises a plurality of integrated devices with each integrated device having a sharp member, and wherein each sharp member has a same payload, a different payload, or a different payload combination from another sharp member of another integrated device.

Embodiment 75. The integrated package of any one of Embodiments 31-74, wherein the sharp member is capable of carrying a genetic material capable of stably integrating into a cell's genome from a list of gRNA comprising crRNA or a tracrRNA capable of complexing with a Cas protein or plasmid expressing Cas protein, TALENs or zinc finger nucleases.

Embodiment 76. A method for operating an integrated package comprising: providing a power source; providing the integrated package comprising at least one integrated device comprising: a membrane portion having a membrane opening; the membrane portion having a first surface on a first side and a second side, the first side opposite the second side, a MEMS portion disposed on the first side of the membrane portion, the MEMS portion having a sharp member disposed on an actuator stage configured as an electrode and a pull-toward electrode disposed on the first surface of the membrane portion substantially parallel to the actuator stage, and a fluidic portion disposed on the second side of the membrane portion; supplying, via the power source, a voltage across the actuator stage and the pull-toward electrode; generating an electrostatic field between the actuator stage and the pull-toward electrode based on the supplied voltage; and actuating the sharp member to move across the membrane opening and into at least a portion of the fluidic cavity due to the generated electrostatic field between the actuator stage and the pull-toward electrode.

Embodiment 77. The method of Embodiment 76, wherein the fluidic portion comprises a fluidic cap forming a fluidic cavity in the fluidic portion, the fluidic cap having a surface thereon, at least one fluidic inlet, and at least one fluidic outlet.

Embodiment 78. The method of any one of Embodiments 76 or 77, further comprising: prior to supplying the voltage across the actuator stage and the pull-toward electrode, flowing a fluidic medium comprising a plurality of particles via the at least one fluidic inlet at a predetermined flow rate.

Embodiment 79. The method of any one of Embodiments 76-78, wherein the second side of the membrane portion comprises one or more capture-site electrodes disposed on a second surface of the membrane portion and adjacent to the membrane opening.

Embodiment 80. The method of any one of Embodiments 76-79, wherein the one or more capture-site electrodes are electrically connected to form a circular capture-site electrode.

Embodiment 81. The method of any one of Embodiments 76-80, wherein two or more capture-site electrodes of the one or more capture-site electrodes are used partially as a phased sensor array.

Embodiment 82. The method of any one of Embodiments 76-81, wherein the one or more capture-site electrodes are a pair of bi-polar electrodes that are disposed across the membrane opening.

Embodiment 83. The method of any one of Embodiments 76-82, wherein the fluidic cap comprises one or more counter-electrodes disposed on the surface of the fluidic cap across from the membrane opening.

Embodiment 84. The method of any one of Embodiments 76-83, further comprising: prior to supplying the voltage across the actuator stage and the pull-toward electrode, supplying, via the power source, an AC voltage across the one or more counter-electrodes and the one or more capture-site electrodes.

Embodiment 85. The method of any one of Embodiments 76-84, further comprising: generating an electric field with a local maximum proximate the membrane opening; tuning an operating frequency of the AC voltage to create a positive dielectrophoretic force on a portion of the plurality of particles; and capturing one or more of the plurality of particles in the fluid medium.

Embodiment 86. The method of any one of Embodiments 76-85, wherein the tuning of the operating frequency of the AC voltage comprises determining a competing effect of dielectrophoresis (DEP) force induced by the applied AC voltage with respect to a hydrodynamic force exerted upon one or more particles of the flowing fluidic medium.

Embodiment 87. The method of any one of Embodiments 76-86, wherein the capturing one or more of the plurality of particles via the DEP force comprises supplying sufficient DEP force to capture the one or more particles proximate to the membrane opening by overcoming the hydrodynamic force on the one or more particles flowing in the fluid medium.

Embodiment 88. The method of any one of Embodiments 76-87, further comprising: adjusting the AC voltage such that the DEP force is tuned to capture a single particle at a single integrated device.

Embodiment 89. The method of any one of Embodiments 76-88, further comprising: interrogating the single particle by the sharp member.

Embodiment 90. The method of any one of Embodiments 76-89, wherein a tip of the sharp member is configured to deliver a payload to the single particle.

Embodiment 91. The method of any one of Embodiments 76-90, wherein the one or more pull-toward electrodes comprises a sheet of electrode material or the one or more pull-toward electrodes disposed adjacent to the membrane opening and/or at least partially surrounding the membrane opening.

Embodiment 92. The method of any one of Embodiments 76-91, wherein the adjacent to the membrane opening comprises the one or more pull-toward electrodes are disposed partially bracketing the membrane opening.

Embodiment 93. The method of any one of Embodiments 76-92, wherein at least partially surrounding the membrane opening comprises surrounding the membrane opening in at least one dimension.

Embodiment 94. The method of any one of Embodiments 76-93, wherein at least partially surrounding the membrane opening comprises substantially enclosing the membrane opening in at least one dimension.

Embodiment 95. The method of any one of Embodiments 76-94, wherein the one or more pull-toward electrodes are electrically connected to form a circular pull-toward electrode.

Embodiment 96. The method of any one of Embodiments 76-95, wherein the second side of the membrane portion comprises at least a portion of one or more capture-site electrodes partially or fully embedded in the second side of the membrane portion and adjacent to the membrane opening.

Embodiment 97. The method of any one of Embodiments 76-96, wherein the one or more capture-site electrodes comprises a circular capture-site electrode geometry or an annular capture-site electrode geometry.

Embodiment 98. The method of any one of Embodiments 76-97, wherein the one or more capture-site electrodes are a pair of bi-polar electrodes that are disposed across the membrane opening.

Embodiment 99. The method of any one of Embodiments 76-98, wherein the one or more counter-electrodes and the one or more capture-site electrodes are configured as one or more electrode pairs.

Embodiment 100. The method of any one of Embodiments 76-99, wherein the one or more electrode pairs are configured to capture one or more particles in the fluid medium.

Embodiment 101. The method of any one of Embodiments 76-100, wherein the one or more particles are captured using dielectrophoretic force.

Embodiment 102. The method of any one of Embodiments 76-101, wherein the dielectrophoretic force is applied perpendicular to the membrane opening.

Embodiment 103. The method of any one of Embodiments 76-102, wherein the actuator stage is suspended within a MEMS cavity of the MEMS portion and supported by two or more actuator arms attached to walls of the MEMS cavity, wherein the two or more actuator arms comprises at least a serpentine pattern or a conductive material comprising at least one of single crystal silicon, polycrystalline silicon, nanocrystalline silicon, amorphous silicon, hydrogenated amorphous silicon, a metal, a metallic alloy, a eutectic, a ceramic, a composite, a polymer, a doped silicon, an allotrope of silicon, an inorganic glassy material or mixture, an inorganic polycrystalline material or mixture, an inorganic single crystalline material or mixture, a ceramic material comprising metal oxides, metalloid oxides, metal or metalloid nitrides, metal or metalloid oxides with nitrogen or other non-metalloid or metal elements, a doped combination of the above materials, any layered stack or structural combination of the above materials.

Embodiment 104. The method of any one of Embodiments 76-103, wherein the actuator stage is suspended a predetermined distance away from the first surface of the membrane portion, and in operation, the actuator stage moves relative to the first surface of the membrane portion.

Embodiment 105. The method of any one of Embodiments 76-104, wherein the MEMS cavity is configured to contain a fluid within the MEMS cavity.

Embodiment 106. The method of any one of Embodiments 76-105, wherein the fluid disposed within the MEMS cavity is miscible with the fluid medium.

Embodiment 107. The method of any one of Embodiments 76-106, wherein the fluid disposed within the MEMS cavity is immiscible with the fluid medium.

Embodiment 108. The method of any one of Embodiments 76-107, wherein a fluid medium flows from the at least one fluidic inlet to the at least one fluidic outlet at a predetermined flow rate.

Embodiment 109. The method of any one of Embodiments 76-108, wherein the sharp member comprises silicon or is coated with one or more materials configured for conjugation of a polynucleotide to the sharp member.

Embodiment 110. The method of any one of Embodiments 76-109, wherein at least a portion of the sharp member is coated with a plurality of gold atoms, and wherein the sharp member coated with gold atoms is capable of being attached to one or more biological molecules.

Embodiment 111. The method of any one of Embodiments 76-110, wherein the sharp member comprises at least one of a Langmuir-Bodgett film, a functionalized glass, germanium, PTFE, polystyrene, gallium arsenide, silver, membrane, nylon, PVP, silicon oxide, metal oxide, or ceramics.

Embodiment 112. The method of any one of Embodiments 76-111, wherein the sharp member comprises one or more bond types including at least one of a covalent bond, an ionic bond, an electrostatic bond, a hydrogen bond, or a van der waals bond or is conjugated to a polynucleotide via a covalent bond, a thiol (—SH) modifier, avidin/biotin coupling chemistry, or a mediating linker molecule from one of polyethylene glycol (PEG) or polyethyleneimine (PEI).

Embodiment 113. The method of any one of Embodiments 76-112, wherein the sharp member is capable of carrying one or more payloads from a list of nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs, exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs.

Embodiment 114. The method of any one of Embodiments 76-113, wherein the integrated package comprises a plurality of integrated devices with each integrated device having a sharp member, and wherein each sharp member has a same payload, a different payload, or a different payload combination from another sharp member of another integrated device.

Embodiment 115. The method of any one of Embodiments 76-114, wherein the sharp member is capable of carrying a genetic material capable of stably integrating into a cell's genome from a list of gRNA comprising crRNA or a tracrRNA capable of complexing with a Cas protein or plasmid expressing Cas protein, TALENs or zinc finger nucleases.

Embodiment 116. A method of operating an integrated device comprising: providing a power source; providing the integrated device comprising: a membrane portion having a membrane opening; the membrane portion having a first side and a second side, wherein the second side of the membrane portion comprises one or more capture-site electrodes disposed thereon, a MEMS portion disposed on the first side of the membrane portion, and a fluidic portion disposed on the second side of the membrane portion, the fluidic portion comprising a fluidic cap forming a fluidic cavity in the fluidic portion, the fluidic cap having a surface thereon, at least one fluidic inlet, at least one fluidic outlet, and one or more counter-electrodes disposed on the surface of the fluidic cap across from the membrane opening; supplying, via the power source, an AC voltage across the one or more counter-electrodes and the one or more capture-site electrodes; and generating an electric field with a local maximum proximate the membrane opening.

Embodiment 117. The method of Embodiment 116, wherein the second side of the membrane portion comprises at least a portion of the one or more capture-site electrodes partially or fully embedded in the second side of the membrane portion and adjacent to the membrane opening.

Embodiment 118. The method of any one of Embodiments 116 or 117, wherein the one or more capture-site electrodes are disposed adjacent to the membrane opening and comprises a circular capture-site electrode geometry or an annular capture-site electrode geometry.

Embodiment 119. The method of any one of Embodiments 116-118, wherein the one or more capture-site electrodes are a pair of bi-polar electrodes that are disposed across the membrane opening.

Embodiment 120. The method of any one of Embodiments 116-119, wherein the one or more counter-electrodes and the one or more capture-site electrodes are configured as one or more electrode pairs.

Embodiment 121. The method of any one of Embodiments 116-120, wherein the MEMS portion comprises a sharp member disposed on an actuator stage within a MEMS cavity.

Embodiment 122. The method of any one of Embodiments 116-121, wherein the first side of the membrane portion comprises one or more pull-toward electrodes disposed on the first surface of the membrane portion and adjacent to the membrane opening and/or at least partially surrounding the membrane opening.

Embodiment 123. The method of any one of Embodiments 116-122, wherein the one or more pull-toward electrodes comprises a sheet of electrode material or the one or more pull-toward electrodes are disposed partially bracketing the membrane opening.

Embodiment 124. The method of any one of Embodiments 116-123, wherein at least partially surrounding the membrane opening comprises surrounding the membrane opening in at least one dimension.

Embodiment 125. The method of any one of Embodiments 116-124, wherein at least partially surrounding the membrane opening comprises substantially enclosing the membrane opening in at least one dimension.

Embodiment 126. The method of any one of Embodiments 116-125, wherein the one or more pull-toward electrodes are electrically connected to form a circular pull-toward electrode.

Embodiment 127. The method of any one of Embodiments 116-126, wherein the actuator stage is configured as an electrode and is substantially parallel to the one or more pull-toward electrodes.

Embodiment 128. The method of any one of Embodiments 116-127, further comprising: prior to supplying the AC voltage across the one or more counter-electrodes and the one or more capture-site electrodes, flowing a fluidic medium comprising a plurality of particles via the at least one fluidic inlet at a predetermined flow rate.

Embodiment 129. The method of any one of Embodiments 116-128, further comprising: tuning an operating frequency of the AC voltage to create a positive dielectrophoretic force on a portion of the plurality of particles; and capturing one or more of the plurality of particles in the fluid medium.

Embodiment 130. The method of any one of Embodiments 116-129, wherein the tuning of the operating frequency of the AC voltage comprises determining a competing effect of dielectrophoresis (DEP) force induced by the applied AC voltage with respect to a hydrodynamic force exerted upon one or more particles of the flowing fluidic medium.

Embodiment 131. The method of any one of Embodiments 116-130, wherein the capturing one or more of the plurality of particles via the DEP force comprises supplying sufficient DEP force to capture the one or more particles proximate to the membrane opening by overcoming the hydrodynamic force on the one or more particles flowing in the fluid medium.

Embodiment 132. The method of any one of Embodiments 116-131, further comprising: adjusting the AC voltage such that the DEP force is tuned to capture a single particle at a single capture site.

Embodiment 133. The method of any one of Embodiments 116-132, further comprising: interrogating the single particle by the sharp member.

Embodiment 134. The method of any one of Embodiments 116-133, wherein a tip of the sharp member is configured to deliver one or more payloads to the single particle.

Embodiment 135. The method of any one of Embodiments 116-134, further comprising: supplying, via the power source, a voltage across the actuator stage and the one or more pull-toward electrodes; generating an electrostatic field between the actuator stage and the one or more pull-toward electrodes based on the supplied voltage; and causing the sharp member to move across the membrane opening and into at least a portion of the fluidic cavity based on the generated electrostatic field between the actuator stage and the one or more pull-toward electrodes.

Embodiment 136. The method of any one of Embodiments 116-135, wherein the one or more captured particles are interrogated by the sharp member when actuated.

Embodiment 137. The method of any one of Embodiments 116-136, wherein a tip of the sharp member is configured to deliver one or more payloads to the one or more captured particles.

Embodiment 138. The method of any one of Embodiments 116-137, wherein the second side of the membrane portion comprises at least a portion of one or more capture-site electrodes partially or fully embedded in the second side of the membrane portion and adjacent to the membrane opening.

Embodiment 139. The method of any one of Embodiments 116-138, wherein the one or more capture-site electrodes comprises a circular capture-site electrode geometry or an annular capture-site electrode geometry.

Embodiment 140. The method of any one of Embodiments 116-139, wherein the one or more capture-site electrodes are a pair of bi-polar electrodes that are disposed across the membrane opening.

Embodiment 141. The method of any one of Embodiments 116-140, wherein the actuator stage is suspended within the MEMS cavity of the MEMS portion and supported by two or more actuator arms attached to walls of the MEMS cavity, wherein the two or more actuator arms comprises at least a serpentine pattern or a conductive material comprising at least one of single crystal silicon, polycrystalline silicon, nanocrystalline silicon, amorphous silicon, hydrogenated amorphous silicon, a metal, a metallic alloy, a eutectic, a ceramic, a composite, a polymer, a doped silicon, an allotrope of silicon, an inorganic glassy material or mixture, an inorganic polycrystalline material or mixture, an inorganic single crystalline material or mixture, a ceramic material comprising metal oxides, metalloid oxides, metal or metalloid nitrides, metal or metalloid oxides with nitrogen or other non-metalloid or metal elements, a doped combination of the above materials, any layered stack or structural combination of the above materials.

Embodiment 142. The method of any one of Embodiments 116-141, wherein the actuator stage is suspended a predetermined distance away from the first surface of the membrane portion, and in operation, the actuator stage moves relative to the first surface of the membrane portion.

Embodiment 143. The method of any one of Embodiments 116-142, wherein the MEMS cavity is configured to contain a fluid within the MEMS cavity.

Embodiment 144. The method of any one of Embodiments 116-143, wherein the fluid disposed within the MEMS cavity is miscible with the fluid medium.

Embodiment 145. The method of any one of Embodiments 116-144, wherein the fluid disposed within the MEMS cavity is immiscible with the fluid medium.

Embodiment 146. The method of any one of Embodiments 116-145, wherein the sharp member comprises silicon.

Embodiment 147. The method of any one of Embodiments 116-146, wherein the sharp member is coated with one or more materials configured for conjugation of a polynucleotide to the sharp member.

Embodiment 148. The method of any one of Embodiments 116-147, wherein at least a portion of the sharp member is coated with a plurality of gold atoms, and wherein the sharp member coated with gold atoms is capable of being attached to one or more biological molecules.

Embodiment 149. The method of any one of Embodiments 116-148, wherein the sharp member comprises at least one of a Langmuir-Bodgett film, a functionalized glass, germanium, PTFE, polystyrene, gallium arsenide, silver, membrane, nylon, PVP, silicon oxide, metal oxide, or ceramics.

Embodiment 150. The method of any one of Embodiments 116-149, wherein the sharp member comprises one or more bond types including at least one of a covalent bond, an ionic bond, an electrostatic bond, a hydrogen bond, or a van der waals bond or is conjugated to a polynucleotide via a covalent bond, a thiol (—SH) modifier, avidin/biotin coupling chemistry, or a mediating linker molecule from one of polyethylene glycol (PEG) or polyethyleneimine (PEI).

Embodiment 151. The method of any one of Embodiments 116-150, wherein the sharp member is capable of carrying one or more payloads from a list of nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs, exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs.

Embodiment 152. The method of any one of Embodiments 116-151, wherein the sharp member is capable of carrying a genetic material capable of stably integrating into a cell's genome from a list of gRNA comprising crRNA or a tracrRNA capable of complexing with a Cas protein or plasmid expressing Cas protein, TALENs or zinc finger nucleases.

Embodiment 153. An integrated device comprising: a membrane portion having a membrane opening; the membrane portion having a first side and a second side, the first side opposite the second side; a MEMS portion disposed on the first side of the membrane portion, the MEMS portion having a sharp member disposed on an actuator stage within a MEMS cavity, the sharp member having a distal (or base) portion attached substantially perpendicular to the actuator stage; and a fluidic portion disposed on the second side of the membrane portion, the fluidic portion having a fluidic cavity for flowing a fluid medium within the fluidic portion, wherein the membrane opening provides access between the MEMS portion and the fluidic portion and is substantially aligned with a proximal portion of the sharp member, and in operation, the proximal portion of the sharp member moves across the membrane opening and into at least a portion of the fluidic cavity.

Embodiment 154. The integrated device of Embodiment 153, wherein the first side of the membrane portion comprises one or more pull-toward electrodes disposed on a first surface of the membrane portion and adjacent to the membrane opening and/or at least partially surrounding the membrane opening.

Embodiment 155. The integrated device of Embodiments 153 or 154, wherein the second side of the membrane portion comprises at least a portion of one or more capture-site electrodes partially or fully embedded in the second side of the membrane portion and adjacent to the membrane opening.

Embodiment 156. The integrated device of any one of Embodiments 153-155, wherein the second side of the membrane portion comprises one or more capture-site electrodes disposed on a second surface of the membrane portion and adjacent to the membrane opening.

Embodiment 157. The integrated device of Embodiment 156, wherein the fluidic portion comprises one or more counter-electrodes disposed on a fluidic cavity surface across from the membrane opening, and in operation, the one or more counter-electrodes and the one or more capture-site electrodes are configured as one or more electrode pairs.

Embodiment 158. The integrated device of Embodiments 156 or 157, wherein the one or more electrode pairs are configured to capture one or more particles in the fluid medium.

Embodiment 159. The integrated device of Embodiment 158, wherein the one or more particles are captured using dielectrophoretic force.

Embodiment 160. The integrated device of any one of Embodiments 153-159, wherein the actuator stage is suspended within the MEMS cavity and supported by two or more actuator arms attached to walls of the MEMS cavity, wherein the two or more actuator arms comprises at least a serpentine pattern or a conductive material comprising at least one of single crystal silicon, polycrystalline silicon, nanocrystalline silicon, amorphous silicon, hydrogenated amorphous silicon, a metal, a metallic alloy, a eutectic, a ceramic, a composite, a polymer, a doped silicon, an allotrope of silicon, an inorganic glassy material or mixture, an inorganic polycrystalline material or mixture, an inorganic single crystalline material or mixture, a ceramic material comprising metal oxides, metalloid oxides, metal or metalloid nitrides, metal or metalloid oxides with nitrogen or other non-metalloid or metal elements, a doped combination of the above materials, any layered stack or structural combination of the above materials.

Embodiment 161. The integrated device of any one of Embodiments 153-160, wherein the sharp member is disposed on a first surface of the actuator stage and the MEMS cavity comprises one or more pull-away electrodes disposed on a MEMS cavity surface facing a second surface of the actuator stage.

Embodiment 162. The integrated device of any one of Embodiments 153-161, wherein the fluidic portion comprises a fluidic inlet and a fluidic outlet and the fluid medium flows from the fluidic inlet to the fluidic outlet at a predetermined flow rate.

Embodiment 163. The integrated device of any one of Embodiments 153-162, wherein the sharp member is capable of carrying one or more payloads from a list of nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs, exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs.

Embodiment 164. An integrated package comprising: a substrate; and a lab-on-chip (LOC) disposed on the substrate, the LOC comprising at least one integrated device comprising: a membrane portion having a membrane opening; the membrane portion having a first side and a second side, the first side opposite the second side, a MEMS portion disposed on the first side of the membrane portion, the MEMS portion having a sharp member disposed on an actuator stage within a MEMS cavity, and a fluidic portion disposed on the second side of the membrane portion, the fluidic portion having a fluidic cavity for flowing a fluid medium within the fluidic portion; and a fluidic cap forming a surface of the fluidic portion of the LOC, the fluidic cap having a fluidic inlet and a fluidic outlet.

Embodiment 165. A method of operating an integrated device comprising: providing a power source; providing the integrated device comprising: a membrane portion having a membrane opening; the membrane portion having a first side and a second side, wherein the second side of the membrane portion comprises one or more capture-site electrodes disposed thereon, a MEMS portion disposed on the first side of the membrane portion, and a fluidic portion disposed on the second side of the membrane portion, the fluidic portion comprising a fluidic cap forming a fluidic cavity in the fluidic portion, the fluidic cap having a surface thereon, at least one fluidic inlet, at least one fluidic outlet, and one or more counter-electrodes disposed on the surface of the fluidic cap across from the membrane opening; supplying, via the power source, an AC voltage across the one or more counter-electrodes and the one or more capture-site electrodes; and generating an electric field with a local maximum proximate the membrane opening.

Embodiment 166. The method of Embodiment 165, further comprising: tuning an operating frequency of the AC voltage to create a positive dielectrophoretic force on a portion of the plurality of particles; and capturing one or more of the plurality of particles in the fluid medium.

Embodiment 167. The method of Embodiment 166, further comprising: supplying, via the power source, a voltage across the actuator stage and the one or more pull-toward electrodes; generating an electrostatic field between the actuator stage and the one or more pull-toward electrodes based on the supplied voltage; and causing the sharp member to move across the membrane opening and into at least a portion of the fluidic cavity based on the generated electrostatic field between the actuator stage and the one or more pull-toward electrodes.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

1. An integrated device comprising:
a membrane portion having a membrane opening; the membrane portion having a first side and a second side, the first side opposite the second side;
a MEMS portion disposed on the first side of the membrane portion, the MEMS portion having a sharp member disposed on an actuator stage within a MEMS cavity, the sharp member having a distal (or base) portion attached substantially perpendicular to the actuator stage; and
a fluidic portion disposed on the second side of the membrane portion, the fluidic portion having a fluidic cavity for flowing a fluid medium within the fluidic portion,
wherein the membrane opening provides access between the MEMS portion and the fluidic portion and is substantially aligned with a proximal portion of the sharp member, and
in operation, the proximal portion of the sharp member moves across the membrane opening and into at least a portion of the fluidic cavity.

2. The integrated device of claim 1, wherein the first side of the membrane portion comprises one or more pull-toward electrodes disposed on a first surface of the membrane portion and adjacent to the membrane opening and/or at least partially surrounding the membrane opening.

3. The integrated device of claim 1, wherein the second side of the membrane portion comprises at least a portion of one or more capture-site electrodes partially or fully embedded in the second side of the membrane portion and adjacent to the membrane opening.

4. The integrated device of claim 1, wherein the second side of the membrane portion comprises one or more capture-site electrodes disposed on a second surface of the membrane portion and adjacent to the membrane opening.

5. The integrated device of claim 4, wherein the fluidic portion comprises one or more counter-electrodes disposed on a fluidic cavity surface across from the membrane opening, and in operation, the one or more counter-electrodes and the one or more capture-site electrodes are configured as one or more electrode pairs.

6. The integrated device of claim 5, wherein the one or more electrode pairs are configured to capture one or more particles in the fluid medium.

7. The integrated device of claim 6, wherein the one or more particles are captured using dielectrophoretic force.

8. The integrated device of claim 1, wherein the actuator stage is suspended within the MEMS cavity and supported by two or more actuator arms attached to walls of the MEMS cavity, wherein the two or more actuator arms comprises at least a serpentine pattern or a conductive material comprising at least one of single crystal silicon, polycrystalline silicon, nanocrystalline silicon, amorphous silicon, hydrogenated amorphous silicon, a metal, a metallic alloy, a eutectic, a ceramic, a composite, a polymer, a doped silicon, an allotrope of silicon, an inorganic glassy material or mixture, an inorganic polycrystalline material or mixture, an inorganic single crystalline material or mixture, a ceramic material comprising metal oxides, metalloid oxides, metal or metalloid nitrides, metal or metalloid oxides with nitrogen or other non-metalloid or metal elements, a doped combination of the above materials, any layered stack or structural combination of the above materials.

9. The integrated device of claim 1, wherein the sharp member is disposed on a first surface of the actuator stage and the MEMS cavity comprises one or more pull-away electrodes disposed on a MEMS cavity surface facing a second surface of the actuator stage.

10. The integrated device of claim 1, wherein the fluidic portion comprises a fluidic inlet and a fluidic outlet and the fluid medium flows from the fluidic inlet to the fluidic outlet at a predetermined flow rate.

11. The integrated device of claim 1, wherein the sharp member is capable of carrying one or more payloads from a list of nucleotide-based molecule, DNA, RNA, a viral DNA, circular nucleotide sequence, linear nucleotide sequence, a single stranded nucleotide, circular DNA, plasmids, linear DNA, a hybrid DNA-RNA molecule, proteins, peptides, metabolites, viruses, capsid nanoparticles, membrane impermeable drugs, exogenous organelles, molecular probes, nanoscale devices, nanoscale sensors, nanoscale probes, nanoscale plasmonic optical switches, carbon nanotubes, quantum dots, nanoparticles, inhibitory antibodies, stimulatory transcription factors including at least one of Oct4 or Sox2, silencing DNA, siRNA, HDAC inhibitors, DNA methyltransferase inhibitors, one or more molecules that increases or decreases gene expression, a protein, antibodies, enzymes, one or more small molecule drugs.

12. An integrated package comprising:
a substrate; and
a lab-on-chip (LOC) disposed on the substrate, the LOC comprising at least one integrated device comprising:
a membrane portion having a membrane opening; the membrane portion having a first side and a second side, the first side opposite the second side,
a MEMS portion disposed on the first side of the membrane portion, the MEMS portion having a sharp member disposed on an actuator stage within a MEMS cavity, and
a fluidic portion disposed on the second side of the membrane portion, the fluidic portion having a fluidic cavity for flowing a fluid medium within the fluidic portion; and
a fluidic cap forming a surface of the fluidic portion of the LOC, the fluidic cap having a fluidic inlet and a fluidic outlet.

13. A method of operating an integrated device comprising:
providing a power source;
providing the integrated device comprising:
a membrane portion having a membrane opening; the membrane portion having a first side and a second side, wherein the second side of the membrane portion comprises one or more capture-site electrodes disposed thereon,
a MEMS portion disposed on the first side of the membrane portion, and
a fluidic portion disposed on the second side of the membrane portion, the fluidic portion comprising a fluidic cap forming a fluidic cavity in the fluidic portion, the fluidic cap having a surface thereon, at least one fluidic inlet, at least one fluidic outlet, and one or more counter-electrodes disposed on the surface of the fluidic cap across from the membrane opening;
supplying, via the power source, an AC voltage across the one or more counter-electrodes and the one or more capture-site electrodes; and
generating an electric field with a local maximum proximate the membrane opening.

14. The method of claim 13, further comprising:
tuning an operating frequency of the AC voltage to create a positive dielectrophoretic (DEP) force on a portion of the plurality of particles; and
capturing one or more of the plurality of particles in the fluid medium.

15. The method of claim 14, wherein the tuning of the operating frequency of the AC voltage comprises determining a competing effect of DEP force induced by the applied AC voltage with respect to a hydrodynamic force exerted upon one or more particles of the flowing fluidic medium.

16. The method of claim 14, wherein the capturing one or more of the plurality of particles via the DEP force comprises supplying sufficient DEP force to capture the one or more particles proximate to the membrane opening by overcoming the hydrodynamic force on the one or more particles flowing in the fluid medium.

17. The method of claim 14, further comprising:
adjusting the AC voltage such that the DEP force is tuned to capture a single particle at a single capture site.

18. The method of claim 17, further comprising:
interrogating the single particle by the sharp member.

19. The method of claim 17, wherein a tip of the sharp member is configured to deliver one or more payloads to the single particle.

20. The method of claim 14, further comprising:
supplying, via the power source, a voltage across the actuator stage and the one or more pull-toward electrodes;
generating an electrostatic field between the actuator stage and the one or more pull-toward electrodes based on the supplied voltage; and
causing the sharp member to move across the membrane opening and into at least a portion of the fluidic cavity based on the generated electrostatic field between the actuator stage and the one or more pull-toward electrodes.
